(19) United States
(12) Patent Application Publication
(10) Pub. No.: US 2023/0389859 A1
(43) Pub. Date: Dec. 7, 2023

(54) INDEX VALUE ESTIMATION DEVICE, ESTIMATION SYSTEM, INDEX VALUE ESTIMATION METHOD, AND RECORDING MEDIUM

(71) Applicant: NEC Corporation, Tokyo (JP)

(72) Inventors: Kazuki IHARA, Tokyo (JP); Kenichiro FUKUSHI, Tokyo (JP); Zhenwei WANG, Tokyo (JP); Chenhui HUANG, Tokyo (JP); Hiroshi KAJITANI, Tokyo (JP); Fumiyuki NIHEY, Tokyo (JP); Yoshitaka NOZAKI, Tokyo (JP); Kentaro NAKAHARA, Tokyo (JP)

(73) Assignee: NEC Corporation, Tokyo (JP)

(21) Appl. No.: 18/202,829

(22) Filed: May 26, 2023

(30) Foreign Application Priority Data
Jun. 6, 2022 (JP) 2022-091434

(51) Int. Cl. A61B 5/00; A61B 5/11

(52) U.S. Cl. CPC A61B 5/4585; A61B 5/112; A61B 5/6807; A61B 2562/0219

(57) ABSTRACT

An index value estimation device including a data acquisition unit that acquires feature amount data including a feature amount extracted from sensor data related to a motion of a foot of a user and used for estimating an index value indicating a knee state of the user, a storage unit that stores an estimation model that outputs an index value according to an input of the feature amount data, an estimation unit that estimates an output obtained by inputting the acquired feature amount data to the estimation model as an index value indicating the knee state of the user, and an output unit that outputs information about the estimated index value indicating the knee state of the user.

This application is based upon and claims the benefit of priority from Japanese Patent Application No. 2022-091434, filed on Jun. 6, 2022, the disclosure of which is incorporated herein in its entirety by reference.

### TECHNICAL FIELD

The present disclosure relates to an index value estimation device or the like that estimates an index value indicating a knee state.

### BACKGROUND ART

With growing interest in healthcare, services that provide information according to a gait have attracted attention. For example, a technique for analyzing a gait using sensor data measured by a sensor mounted in footwear such as shoes has been developed. A feature associated with a gait event related to a physical condition appears in the time series data of the sensor data. The physical condition of the subject can be estimated by analyzing the gait data including the feature associated with the gait event. For example, when the condition of the knee of the subject can be estimated, early detection and prevention of diseases such as knee osteoarthritis can be performed.

Patent Literature 1 (JP 2016-106948 A) discloses a knee state determination system that determines a knee state of a user by focusing on a motion of a knee region during stepping. The system of Patent Literature 1 includes a plurality of sensor devices and a knee state estimation device. The plurality of sensor devices are attached to each of the waist, the thighs of both legs, and the lower legs of both legs. The plurality of sensor devices measure angular velocities generated by turning motions of the thigh and the lower leg accompanying the stepping of the user. The plurality of sensor devices transmit a turning angular velocity reflecting the measured angular velocity to the knee state determination device. The knee state determination device analyzes data transmitted from the sensor devices to determine the knee state of the user. Specifically, the knee state determination device determines abnormality of the knee of the user using the yaw direction component around the axis in the gravity direction output from each sensor device attached to the thighs and the lower thighs of both legs.

Patent Literature 2 (JP 2022-051451 A) discloses a detection device used for estimating a state during the motion. The devices of JP 2022-051451 A include sensors such as an acceleration sensor and an angular velocity sensor. The devices of Patent Literature 2 are attached to a knee and around a knee of a subject. The acceleration detected by the device of Patent Literature 2 is used to estimate the knee state. Patent Literature 2 discloses estimating the degree and prognosis of knee osteoarthritis using the detected acceleration.
Non-Patent Literature 1 (Yuki Ishikawa et al., “The Method of Diagnosis for the Knee Joint Disease with Individual Modeling—To Clarify the Mechanism of Knee Osteoarthritis”, Proceedings of the 2012 JSME Conference on Robotics and Mechatronics, Hamamatsu, Japan, May 27-29, (2012), pp. 2P1-I02 (1)-2 P1-I02 (2)) reports construction of a knee disease diagnosis method such as knee osteoarthritis. Non-Patent Literature 1 discloses a height, a leg length, a range of a motion of a joint, and lower limb alignment as factors that affect a gait pattern.
Non-Patent Literature 2 (Takashi Komura et al., “Gait Analysis of the Patients with Varus Knee Osteoarthritis”, Journal of the Medical School of Kobe University, 61(4), (2001), pp. 89-94) reports a verification result regarding gait analysis performed on a plurality of subjects for the purpose of quantitatively evaluating lateral thrust observed in patients with knee osteoarthritis.
Non-Patent Literature 3 (Shunsuke Yamashina, “Development of Gait Abnormality Evaluation Method by Observation in Patients with Knee Osteoarthritis Under Conservative Therapy and Verification of Relationship with Reduction in Physical Activity Amount”, Doctoral thesis of Kibi International University, 2019) reports a result of evaluating gait abnormality caused by knee osteoarthritis in a plurality of subjects.
Non-Patent Literature 4 (S. R. Goldberg, et al., “Muscles that influence knee flexion velocity in double support: implications for stiff-knee gait”, Journal of Biomechanics, 37, (2004), pp. 1189-1196) reports a result of examining a muscle that affects a knee bending speed in a both-leg support period during a gait. Non-Patent Literature 4 describes that when the knee bending speed is sufficient in the toe off, proper knee bending can be obtained in the swing phase.

The method of Patent Literature 1 includes estimating the knee state of the user using angular velocities measured by a plurality of sensors attached to the body. In the method of Patent Literature 1, it is necessary to attach sensors to a plurality of positions of the waist and the legs. Therefore, it is difficult to apply the method of Patent Literature 1 to the application of estimating the knee state of the user in daily life.

The technique of Patent Literature 2 discloses estimating the knee state using acceleration or angular velocity measured by an acceleration sensor attached to the knee or around the knee of the subject. In the method of Patent Literature 2, a sensor is attached to the knee or around the knee with an auxiliary tool having flexibility. Therefore, in the method of Patent Literature 2, since the mounting position of the sensor is likely to change every day, it is difficult to apply the method to an application of appropriately estimating the knee state of the user.

When large-scale equipment is used as in Non-Patent Literature 1 to 4, disease such as knee osteoarthritis can be verified in detail. However, since the method as in Non-Patent Literature 1 to 4 requires large-scale equipment, it is difficult to apply the methods to an application of estimating a knee condition in daily life.

An object of the present disclosure is to provide an index value estimation device and the like capable of appropriately estimating an index value indicating a knee state in daily life.

### SUMMARY

An index value estimation device according to an aspect of the present disclosure includes a data acquisition unit that acquires feature amount data including a feature amount to be used for estimating an index value indicating a knee state of a user, the feature amount being extracted from sensor data related to a motion of a foot of the user, a storage unit that stores an estimation model that outputs an index value according to an input of the feature amount data, an estimation unit that estimates, as the index value indicating a knee state of the user, an output obtained by inputting the acquired feature amount data to the estimation model, and an output unit that outputs information related to the estimated index value indicating the knee state of the user.

An index value estimation method according to an aspect of the present disclosure includes acquiring feature amount data including a feature amount to be used for estimating an index value indicating a knee state of a user, the feature amount being extracted from sensor data related to a motion of a foot of the user, inputting the acquired feature amount data to an estimation model, the estimation model outputting an index value according to an input of the feature amount data, estimating, as the index value indicating a knee state of the user, an output obtained by inputting the acquired feature amount data to the estimation model, and outputting information related to the estimated index value indicating the knee state of the user.

A program according to an aspect of the present disclosure causes a computer to execute a step of acquiring feature amount data including a feature amount to be used for estimating an index value indicating a knee state of a user, the feature amount being extracted from sensor data related to a motion of a foot of the user, a step of inputting the acquired feature amount data to an estimation model, the estimation model outputting an index value according to an input of the feature amount data, a step of estimating, as the index value indicating a knee state of the user, an output obtained by inputting the acquired feature amount data to the estimation model, and a step of outputting information related to the estimated index value indicating the knee state of the user.

### BRIEF DESCRIPTION OF THE DRAWINGS

Exemplary features and advantages of the present invention will become apparent from the following detailed description when taken with the accompanying drawings in which:

FIG. 1 is a block diagram illustrating an example of a configuration of an estimation system according to a first example embodiment;

FIG. 2 is a block diagram illustrating an example of a configuration of a measurement device included in the estimation system according to the first example embodiment;

FIG. 3 is a conceptual diagram illustrating an arrangement example of the measurement device according to the first example embodiment;

FIG. 4 is a conceptual diagram for describing an example of a relationship between a local coordinate system and a world coordinate system set in the measurement device according to the first example embodiment;

FIG. 5 is a conceptual diagram for describing a human body surface used in the description of the measurement device according to the first example embodiment;

FIG. 6 is a conceptual diagram for explaining a gait cycle used in the description regarding the measurement device according to the first example embodiment;

FIG. 7 is a graph for explaining an example of time series data of sensor data measured by the measurement device according to the first example embodiment;

FIG. 8 is a diagram for explaining an example of normalization of gait waveform data extracted from time series data of sensor data measured by the measurement device according to the first example embodiment;

FIG. 9 is a block diagram illustrating an example of a configuration of an index value estimation device included in the estimation system according to the first example embodiment;

FIG. 10 is a graph for explaining a parameter regarding a knee flexion angle estimated by the estimation system according to the first example embodiment;

FIG. 11 is a table summarizing an example of feature amounts used for estimating parameters regarding a knee flexion angle estimated by the estimation system according to the first example embodiment;

FIG. 12 is a table summarizing an example of feature amounts used for estimating parameters regarding a knee flexion angle estimated by the estimation system according to the first example embodiment;

FIG. 13 is a table summarizing an example of feature amounts used for estimating parameters regarding a knee flexion angle estimated by the estimation system according to the first example embodiment;

FIG. 14 is a table summarizing an example of feature amounts used for estimating parameters regarding a knee flexion angle estimated by the estimation system according to the first example embodiment;

FIG. 15 is a table summarizing an example of feature amounts used for estimating parameters regarding a knee flexion angle estimated by the estimation system according to the first example embodiment;

FIG. 16 is a flowchart for explaining an example of the operation of the measurement device included in the estimation system according to the first example embodiment;

FIG. 17 is a flowchart for explaining an example of the operation of the index value estimation device included in the estimation system according to the first example embodiment;

FIG. 18 is a conceptual diagram for describing an application example of the estimation system according to the first example embodiment;

FIG. 19 is a block diagram illustrating an example of a configuration of an estimation system according to the second example embodiment;

FIG. 20 is a graph for explaining Angular Jerk Cost estimated by the estimation system according to a second example embodiment;

FIG. 21 is a flowchart for explaining an example of the operation of a measurement device included in the estimation system according to the second example embodiment;

FIG. 22 is a flowchart for explaining an example of the operation of the index value estimation device included in the estimation system according to the second example embodiment;

FIG. 23 is a conceptual diagram for describing an application example of the estimation system according to the second example embodiment;

FIG. 24 is a block diagram illustrating an example of a configuration of an index value estimation device according to a third example embodiment; and

FIG. 25 is a block diagram illustrating an example of a hardware configuration that executes control and processing according to each example embodiment.

### EXAMPLE EMBODIMENT

Example embodiments of the present invention will be described below with reference to the drawings. In the following example embodiments, technically preferable limitations are imposed to carry out the present invention, but the scope of this invention is not limited to the following description. In all drawings used to describe the following example embodiments, the same reference numerals denote similar parts unless otherwise specified. In addition, in the following example embodiments, a repetitive description of similar configurations or arrangements and operations may be omitted.

### First Example Embodiment

First, an estimation system according to a first example embodiment will be described with reference to the drawings. The estimation system according to the present example embodiment measures sensor data related to a motion of a foot according to a gait of a user. The estimation system according to the present example embodiment estimates an index value indicating the knee state of the user using the measured sensor data. In the present example embodiment, an example of estimating a parameter related to a knee flexion angle as an index value indicating a knee state will be described. The knee flexion angle is an angle formed by the thigh and the lower leg around the knee joint. In the present example embodiment, the knee flexion angle indicates an angle in a plane in the traveling direction (in the sagittal plane).

(Configuration)

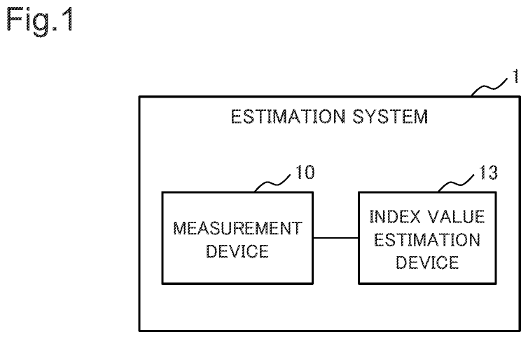

FIG. 1 is a block diagram illustrating an example of a configuration of an estimation system 1 according to the present example embodiment. The estimation system 1 includes a measurement device 10 and an index value estimation device 13. In the present example embodiment, an example in which the measurement device 10 and the index value estimation device 13 are configured as separate hardware will be described. For example, the measurement device 10 is installed in footwear or the like of a subject (user) for whom an index value indicating a knee state is to be estimated. For example, the function of the index value estimation device 13 is installed in a mobile terminal carried by the subject (user). Hereinafter, configurations of the measurement device 10 and the index value estimation device 13 will be individually described.

### [Measurement Device]

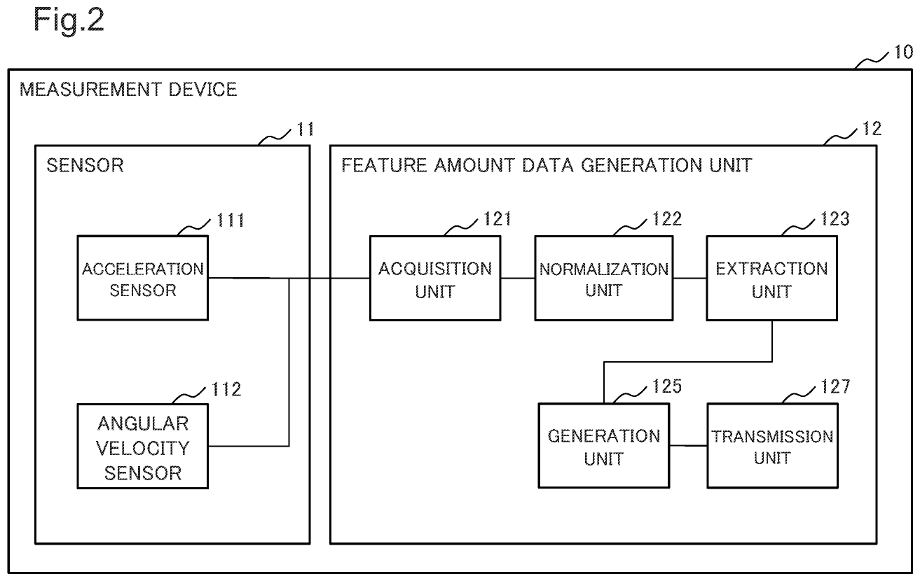

FIG. 2 is a block diagram illustrating an example of a configuration of the measurement device 10. The measurement device 10 includes a sensor 11 and a feature amount data generation unit 12. In the present example embodiment, an example in which the sensor 11 and the feature amount data generation unit 12 are integrated will be described. The sensor 11 and the feature amount data generation unit 12 may be provided as separate devices. For example, the feature amount data generation unit 12 may be incorporated in the index value estimation device 13. In this case, the measurement device 10 transmits the sensor data measured by the sensor 11 to the index value estimation device 13.

As illustrated in FIG. 2, the sensor 11 includes an acceleration sensor 111 and an angular velocity sensor 112. FIG. 2 illustrates an example in which the acceleration sensor 111 and the angular velocity sensor 112 are included in the sensor 11. The sensor 11 may include a sensor other than the acceleration sensor 111 and the angular velocity sensor 112. The sensor, other than the acceleration sensor 111 and the angular velocity sensor 112, that can be included in the sensor 11 will not be described.

The acceleration sensor 111 is a sensor that measures acceleration (also referred to as spatial acceleration) in three axial directions. The acceleration sensor 111 measures acceleration (also referred to as spatial acceleration) as a physical quantity related to the motion of the foot. The acceleration sensor 111 outputs the measured acceleration to the feature amount data generation unit 12. For example, a sensor of a piezoelectric type, a piezoresistive type, a capacitance type, or the like can be used as the acceleration sensor 111. As long as the sensor used as the acceleration sensor 111 can measure acceleration, the measurement method is not limited.

The angular velocity sensor 112 is a sensor that measures angular velocities in three axial directions (also referred to as spatial angular velocities). The angular velocity sensor 112 measures an angular velocity (also referred to as a spatial angular velocity) as a physical quantity related to the motion of the foot. The angular velocity sensor 112 outputs the measured angular velocity to the feature amount data generation unit 12. For example, a sensor of a vibration type, a capacitance type, or the like can be used as the angular velocity sensor 112. As long as the sensor used as the angular velocity sensor 112 can measure the angular velocity, the measurement method is not limited.

The sensor 11 is achieved by, for example, an inertial measurement device that measures acceleration and angular velocity. An example of the inertial measurement device is an inertial measurement unit (IMU). The IMU includes the acceleration sensor 111 that measures acceleration in three axis directions and the angular velocity sensor 112 that measures angular velocities around the three axes. The sensor 11 may be achieved by an inertial measurement device such as a vertical gyro (VG) or an attitude heading reference system (AHRS). The sensor 11 may be achieved by a global positioning system/inertial navigation system (GPS/INS). The sensor 11 may be achieved by a device other than the inertial measurement device as long as it can measure a physical quantity related to the motion of the foot.

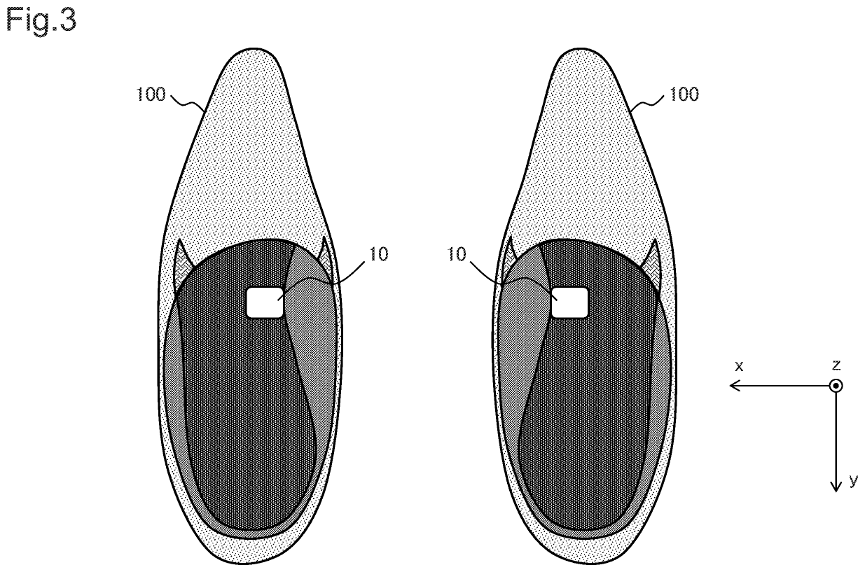

FIG. 3 is a conceptual diagram illustrating an example in which the measurement device 10 is disposed in the shoe 100 of each of both legs. In the example of FIG. 3, the measurement device 10 is installed at a position related to the back side of the arch of foot. For example, the measurement device 10 is disposed in an insole inserted into the shoe 100. For example, the measurement device 10 may be disposed on the bottom face of the shoe 100. For example, the measurement device 10 may be embedded in the main body of the shoe 100. The measurement device 10 may be detachable from the shoe 100 or may not be detachable from the shoe 100. The measurement device 10 may be installed at a position other than the back side of the arch of the foot as long as the sensor data related to the motion of the foot can be measured. The measurement device 10 may be installed on a sock worn by the user or a decorative article such as an anklet worn by the user. The measurement device 10 may be directly attached to the foot or may be embedded in the foot. FIG. 3 illustrates an example in which the measurement devices 10 are installed in the shoe 100 of each of both legs. The measurement device 10 may be installed in the shoe 100 of one foot.

In the example of FIG. 3, a local coordinate system including an x axis in the horizontal direction, a y axis in the front-rear direction, and a z axis in the vertical direction is set with the measurement device 10 (sensor 11) as a reference. In the x axis, the left direction is positive, in the y axis, the rear direction is positive, and in the z axis, the upper direction is positive. The orientation of the axis set in the sensor 11 may be the same for both legs or may be different for both legs. For example, in a case where the sensors 11 produced with the same specifications are disposed in the shoes 100 of both legs, the vertical directions (directions in the Z axis direction) of the sensors 11 disposed in the shoes 100 of both legs are the same. In this case, the three axes of the local coordinate system set in the sensor data derived from the left foot and the three axes of the local coordinate system set in the sensor data derived from the right foot are the same for the left and right feet.

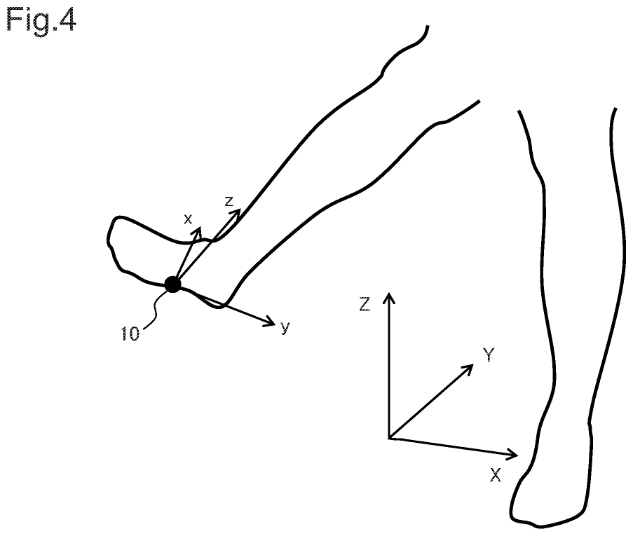

FIG. 4 is a conceptual diagram for describing a local coordinate system (x axis, y axis, z axis) set in the measurement device 10 (sensor 11) installed on the back side of the arch of foot and a world coordinate system (X axis, Y axis, Z axis) set with respect to the ground. In the world coordinate system (X axis, Y axis, Z axis), in a state where the user facing the traveling direction is standing upright, a lateral direction of the user is set to an X-axis direction (leftward direction is positive), a back face direction of the user is set to a Y-axis direction (rearward direction is positive), and a gravity direction is set to a Z-axis direction (vertically upward direction is positive). The example of FIG. 4 conceptually illustrates the relationship between the local coordinate system (x axis, y axis, z axis) and the world coordinate system (X axis, Y axis, Z axis), and does not accurately illustrate the relationship between the local coordinate system and the world coordinate system that varies depending on the gait of the user.

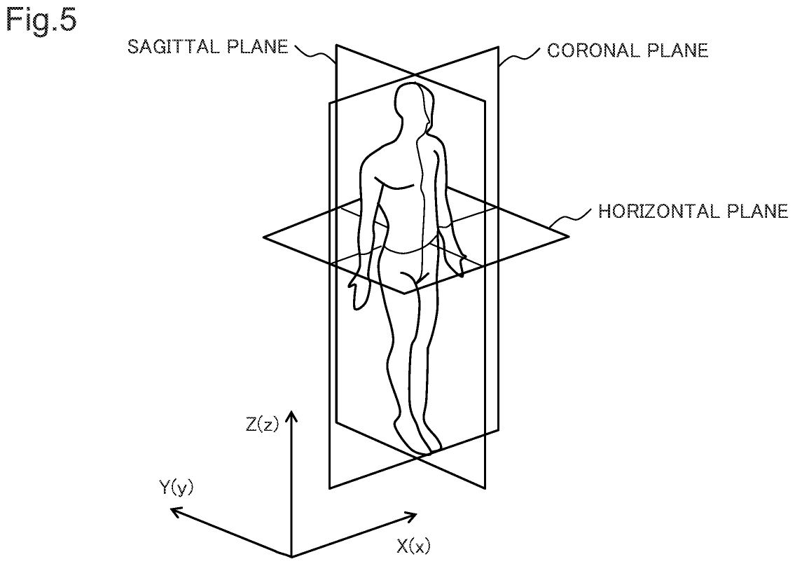

FIG. 5 is a conceptual diagram for describing a face (also referred to as a human body surface) set for the human body. In the present example embodiment, a sagittal plane dividing the body into left and right, a coronal plane dividing the body into front and rear, and a horizontal plane dividing the body horizontally are defined. As illustrated in FIG. 5, the world coordinate system and the local coordinate system coincide with each other in a state in which the user is standing upright with the center line of the foot being directed in the traveling direction. In the present example embodiment, rotation in the sagittal plane with the x axis as a rotation axis is defined as roll, rotation in the coronal plane with the y axis as a rotation axis is defined as pitch, and rotation in the horizontal plane with the z axis as a rotation axis is defined as yaw. A rotation angle in a sagittal plane with the x axis as a rotation axis is defined as a roll angle, a rotation angle in a coronal plane with the y axis as a rotation axis is defined as a pitch angle, and a rotation angle in a horizontal plane with the z axis as a rotation axis is defined as a yaw angle.

As illustrated in FIG. 2, the feature amount data generation unit 12 (also referred to as a feature amount data generation device) includes an acquisition unit 121, a normalization unit 122, an extraction unit 123, a generation unit 125, and a transmission unit 127. For example, the feature amount data generation unit 12 is achieved by a microcomputer or a microcontroller that performs overall control and data processing of the measurement device 10. For example, the feature amount data generation unit 12 includes a central processing unit (CPU), a random access memory (RAM), a read only memory (ROM), a flash memory, and the like. The feature amount data generation unit 12 controls the acceleration sensor 111 and the angular velocity sensor 112 to measure the angular velocity and the acceleration. For example, the feature amount data generation unit 12 may be attached to a mobile terminal (not illustrated) carried by a subject (user).

The acquisition unit 121 acquires acceleration in three axial directions from the acceleration sensor 111. The acquisition unit 121 acquires angular velocities around three axes from the angular velocity sensor 112. For example, the acquisition unit 121 performs analog-to-digital conversion (AD conversion) on the acquired physical quantities (analog data) such as angular velocity and acceleration. The physical quantity (analog data) measured by each of the acceleration sensor 111 and the angular velocity sensor 112 may be converted into digital data in each of the acceleration sensor 111 and the angular velocity sensor 112. The acquisition unit 121 outputs the converted digital data (also referred to as sensor data) to the normalization unit 122. The acquisition unit 121 may be configured to store the sensor data in a storage unit (not illustrated). The sensor data includes at least acceleration data converted into digital data and angular velocity data converted into digital data. The acceleration data includes acceleration vectors in three axial directions. The angular velocity data includes angular velocity vectors around three axes. The acceleration data and the angular velocity data are associated with acquisition time of the data. The acquisition unit 121 may add correction such as a mounting error, temperature correction, and linearity correction to the acceleration data and the angular velocity data.

The normalization unit 122 acquires sensor data from the acquisition unit 121. The normalization unit 122 extracts time series data (also referred to as gait waveform data) for one gait cycle from the time series data of the acceleration in the three-axis direction and the angular velocities around the three axes included in the sensor data.

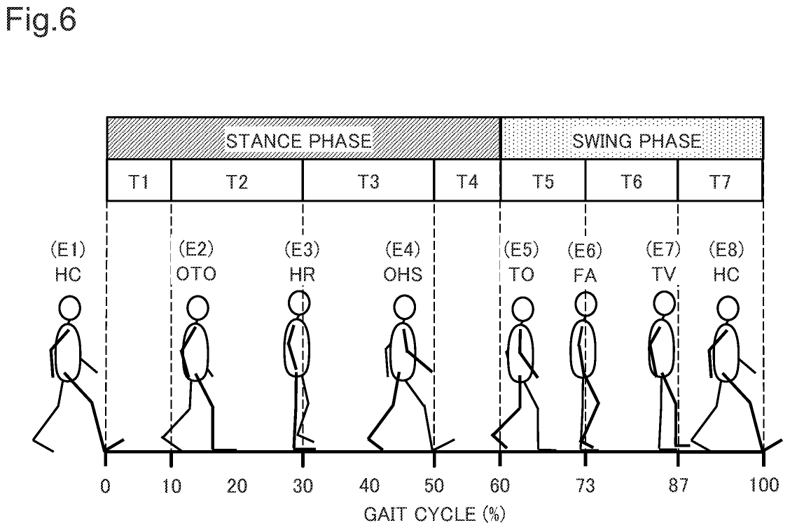

FIG. 6 is a conceptual diagram for explaining a gait event detected in one gait cycle with the right foot as a reference. The horizontal axis of FIG. 6 is a gait cycle normalized with one gait cycle of the right foot as 100%. A time point at which the heel of the right foot lands on the ground is defined as a starting point (0%), and a time point at which the heel of the right foot lands next on the ground is defined as an end point (100%). Each of the plurality of timings included in one gait cycle is a gait phase. One gait cycle of one foot is roughly divided into a stance phase and a swing phase. In the example of FIG. 6, the gait cycle is normalized in such a way that the stance phase occupies 60% and the swing phase occupies 40%. The stance phase is subdivided into an initial stance period T1, a mid-stance period T2 of standing, a terminal stance period T3 of standing, and a pre-swing period T4. The swing phase is subdivided into an initial swing period T5, a mid-swing period T6, and a terminal swing period T7. In the gait waveform in one gait cycle, the time point when the heel lands on the ground may not be set as a starting point. For example, the starting point of the gait waveform in one gait cycle may be set at a center time point of the stance phase or the like.

A gait event E1 represents a heel contact (HC) at the beginning of one gait cycle. The heel contact is an event in which the heel of the right foot, which has been away from the ground in the swing phase, lands on the ground. A gait event E2 represents an opposite toe off (ONO). The opposite toe off is an event in which the toe of the left foot is away from the ground in a state where the ground contact surface of the sole of the right foot is in contact with the ground. A gait event E3 represents a heel rise (HR). The heel rise is an event in which the heel of the right foot is raised in a state where the ground contact surface of the sole of the right foot is in contact with the ground. A gait event E4 represents an opposite heel contact (OHC). The opposite heel contact is an event in which the heel of the left foot, which has been away from the ground in the swing phase of the left foot, lands on the ground. A gait event E5 represents a toe off (TO). The toe off is an event in which the toe of the right foot is away from the ground in a state where the ground contact surface of the sole of the left foot is in contact with the ground. A gait event E6 represents a foot adjacent (FA). The foot adjacent is an event in which the left foot and the right foot cross each other in a state where the ground contact surface of the sole of the left foot is in contact with the ground. A gait event E7 represents a tibia vertical (TV). The tibia vertical is an event in which the tibia of the right foot is substantially perpendicular to the ground while the sole of the left foot is in contact with the ground. A gait event E8 represents a heel strike (HS) at the end of one gait cycle. The gait event E8 corresponds to the end point of the gait cycle starting from the gait event E1 and corresponds to the starting point of the next gait cycle.

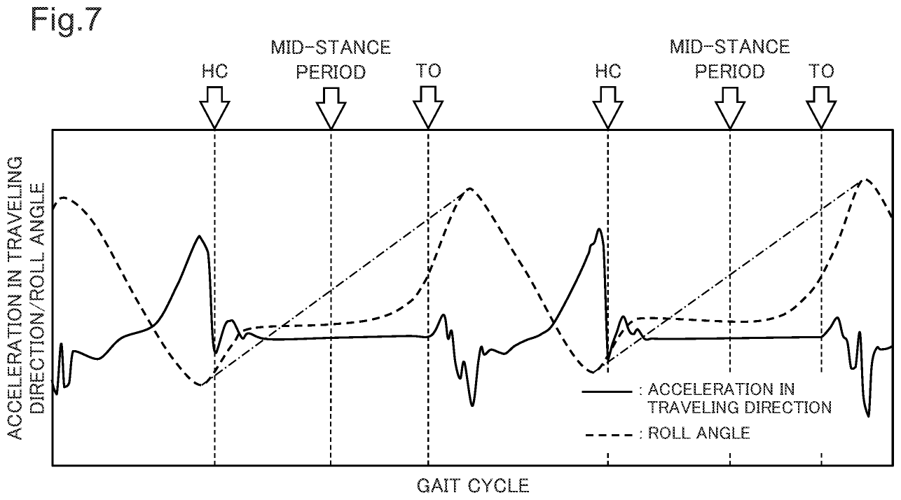

FIG. 7 is a diagram for describing an example of detecting the heel contact HC and the toe off TO from the time series data (solid line) of the acceleration in the traveling direction (Y direction acceleration). The timing of the heel contact HC is the timing of the minimum peak immediately after the maximum peak appearing in the time series data of the acceleration in the traveling direction (Y direction acceleration). The maximum peak serving as a mark of the timing of the heel contact HC corresponds to the maximum peak of the gait waveform data for one gait cycle. A section between the consecutive heel contacts HC is one gait cycle. The timing of the toe off TO is the rising timing of the maximum peak appearing after the period of the stance phase in which the fluctuation does not appear in the time series data of the acceleration in the traveling direction (Y direction acceleration). FIG. 7 also illustrates time series data (broken line) of the roll angle (angular velocity around the X axis). The timing at the midpoint between the timing at which the roll angle is minimum and the timing at which the roll angle is maximum corresponds to the mid-stance period. For example, parameters (also referred to as gait parameters) such as the gait speed, the stride length, the circumduction, the incycloduction/excycloduction, and the plantarflexion/dorsiflexion can also be obtained with the mid-stance period as a reference.

The normalization unit 122 normalizes (also referred to as first normalization) the time of the extracted gait waveform data for one gait cycle to a gait cycle of 0 to 100% (percent). Timing such as 1% or 10% included in the 0 to 100% gait cycle is also referred to as a gait phase. The normalization unit 122 normalizes (also referred to as second normalization) the first normalized gait waveform data for one gait cycle in such a way that the stance phase is 60% and the swing phase is 40%. The stance phase is a period in which at least part of the back side of the foot is in contact with the ground. The swing phase is a period in which the back side of the foot is away from the ground. By performing the second normalization on the gait waveform data, it is possible to reduce the shift of the gait phase from which the feature amount is extracted.

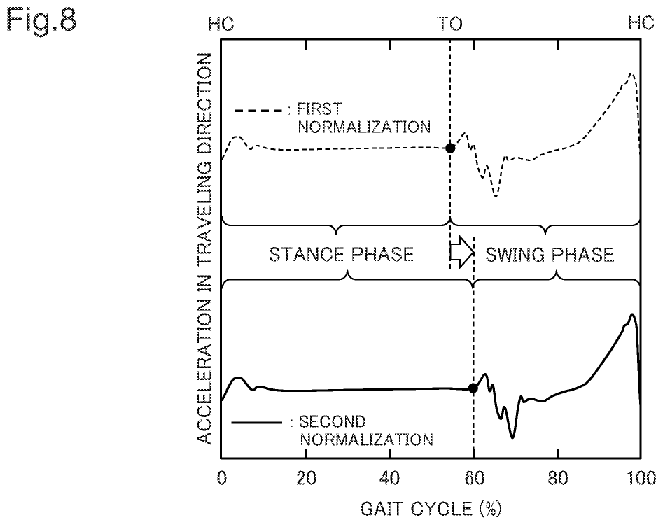

FIG. 8 is a diagram for explaining an example of the gait waveform data normalized by the normalization unit 122. The normalization unit 122 detects the heel contact HC and the toe off TO from the time series data of the acceleration in the traveling direction (Y direction acceleration). The normalization unit 122 extracts a section between consecutive heel contacts HC as gait waveform data for one gait cycle. The normalization unit 122 converts the horizontal axis (time axis) of the gait waveform data for one gait cycle into a gait cycle of 0 to 100% by the first normalization. In FIG. 8, the gait waveform data after the first normalization is indicated by a broken line. In the gait waveform data (broken line) after the first normalization, the timing of the toe off TO is shifted from 60%.

In the example of FIG. 8, the normalization unit 122 normalizes a section from the heel contact HC in which the gait phase is 0% to the toe off TO subsequent to the heel contact HC to 0 to 60%. The normalization unit 122 normalizes a section from the toe off TO to the heel contact HC in which the gait phase subsequent to the toe off TO is 100% to 60 to 100%. As a result, the gait waveform data for one gait cycle is normalized to a section (stance phase) in which the gait cycle is 0 to 60% and a section (swing phase) in which the gait cycle is 60 to 100%. In FIG. 8, the gait waveform data after the second normalization is indicated by a solid line. In the gait waveform data (solid line) after the second normalization, the timing of the toe off TO coincides with 60%.

FIGS. 7 to 8 illustrate examples in which the gait waveform data for one gait cycle is extracted/normalized based on the acceleration in the traveling direction (Y direction acceleration). With respect to acceleration/angular velocity other than the acceleration in the traveling direction (Y direction acceleration), the normalization unit 122 extracts/normalizes gait waveform data for one gait cycle in accordance with the gait cycle of the acceleration in the traveling direction (Y direction acceleration). The normalization unit 122 may generate time series data of angles around three axes by integrating time series data of angular velocities around the three axes. In this case, the normalization unit 122 extracts/normalizes the gait waveform data for one gait cycle in accordance with the gait cycle of the acceleration in the traveling direction (Y direction acceleration) with respect to the angles around the three axes.

The normalization unit 122 may extract/normalize the gait waveform data for one gait cycle based on acceleration/angular velocity other than the acceleration in the traveling direction (Y direction acceleration) (not illustrated). For example, the normalization unit 122 may detect the heel contact HC and the toe off TO from the time series data of the vertical acceleration (Z direction acceleration). The timing of the heel contact HC is a timing of a steep minimum peak appearing in the time series data of the vertical acceleration (Z direction acceleration). At the timing of the steep minimum peak, the value of the vertical acceleration (Z direction acceleration) is substantially zero. The minimum peak serving as a mark of the timing of the heel contact HC corresponds to the minimum peak of the gait waveform data for one gait cycle. A section between the consecutive heel contacts HC is one gait cycle. The timing of the toe off TO is a timing of an inflection point in the middle of gradually increasing after the time series data of the vertical acceleration (Z direction acceleration) passes through a section with a small fluctuation after the maximum peak immediately after the heel contact HC. The normalization unit 122 may extract/normalize the gait waveform data for one gait cycle based on both the acceleration in the traveling direction (Y direction acceleration) and the vertical acceleration (Z direction acceleration). The normalization unit 122 may extract/normalize the gait waveform data for one gait cycle based on acceleration, angular velocity, angle, and the like other than the acceleration in the traveling direction (Y direction acceleration) and the vertical acceleration (Z direction acceleration).

The extraction unit 123 acquires gait waveform data for one gait cycle normalized by the normalization unit 122. The extraction unit 123 extracts a feature amount used for estimating an index value indicating the knee state from the gait waveform data for one gait cycle. For example, the extraction unit 123 extracts a feature amount for each gait phase cluster from a gait phase cluster obtained by integrating temporally continuous gait phases based on a preset condition. The gait phase cluster includes at least one gait phase. The gait phase cluster also includes a single gait phase. The gait waveform data and the gait phase from which the feature amount used to estimate the index value indicating the knee state is extracted will be described later.

The generation unit 125 acquires a feature amount (first feature amount) extracted from each of the gait phases constituting the gait phase cluster. The generation unit 125 applies the feature amount constitutive expression to the acquired first feature amount to generate a feature amount (second feature amount) for each gait phase cluster. The feature amount constitutive expression is a preset calculation expression for generating the feature amount (second feature amount) for each gait phase cluster. For example, the feature amount constitutive expression is a calculation expression related to four arithmetic operations. For example, the second feature amount calculated using the feature amount constitutive expression is an integral average value, an arithmetic average value, an inclination, a variation, or the like of the first feature amount in each gait phase included in the gait phase cluster. For example, the extraction unit 123 applies a calculation expression for calculating the inclination and the variation of the first feature amount extracted from each of the gait phases constituting the gait phase cluster as the feature amount constitutive expression. For example, in a case where the gait phase cluster is configured by a single gait phase, it is not possible to calculate the inclination and the variation, and thus, it is sufficient to use a feature amount constitutive expression for calculating an integral average value, an arithmetic average value, or the like.

The generation unit 125 calculates a parameter (also referred to as a gait parameter) regarding the gait. The generation unit 125 calculates the gait parameter using the feature amount derived from the gait waveform data. For example, the generation unit 125 calculates, as the gait parameters, a stride length, a maximum value of dorsiflexion (maximum dorsiflexion), a ratio of a stance phase in one gait cycle, a ratio of a swing phase in one gait cycle, a maximum value of a toe height (maximum toe height), and a stride time. The gait parameter may be calculated by the index value estimation device 13.

The stride length corresponds to a movement distance in the horizontal plane in a section from a timing of heel contact, which is the start point of one gait cycle to a timing of heel contact, which is the end point. For example, the generation unit 125 calculates, as the stride length, a distance between the starting point and the end point of the trajectory in the horizontal plane obtained by performing second-order integration on the spatial acceleration. The maximum value of dorsiflexion (maximum dorsiflexion) corresponds to the maximum value of the angle of the sole with respect to the horizontal plane. For example, generation unit 125 calculates the spatial angle obtained by integrating the spatial angular velocity as the maximum dorsiflexion. The ratio of the stance phase is a value obtained by dividing a period from a timing of the heel contact which is the starting point of one gait cycle to a timing of the toe off by a period of one gait cycle. In the case of the second normalization, the ratio of the stance phase is 0.6 (60%). The ratio of the swing phase is a value obtained by dividing a period from a timing of the toe off to a timing of the heel contact, which is the end point of one gait cycle, by a period of one gait cycle. In the case of the second normalization, the ratio of the swing phase is 0.4 (40%). The maximum value of the toe height (maximum toe height) is the maximum value of the height in the vertical direction. For example, the generation unit 125 calculates, as the maximum toe height, the maximum value of the vertical height obtained by performing the second-order integration on the vertical acceleration. The stride time corresponds to a time from the timing of the heel contact, which is the start point of one gait cycle, to the timing of the heel contact, which is the end point. For example, the generation unit 125 calculates the stride time by dividing the stride length by the average value (average gait speed) of the traveling direction speed in one gait cycle obtained by integrating the acceleration in the traveling direction. The above-described calculation method is an example, and does not limit the method of calculating the gait parameter.

The transmission unit 127 outputs the feature amount data for each gait phase cluster generated by the generation unit 125. The transmission unit 127 transmits the generated feature amount data of the gait phase cluster to the index value estimation device 13 using the feature amount data. For example, the transmission unit 127 transmits the feature amount data to a data relay device 15 via wireless communication. For example, the transmission unit 127 is configured to transmit the feature amount data to the data relay device 15 via a wireless communication function (not illustrated) conforming to a standard such as Bluetooth (registered trademark) or WiFi (registered trademark). The communication function of the transmission unit 127 may conform to a standard other than Bluetooth (registered trademark) or WiFi (registered trademark).

### [Index Value Estimation Device]

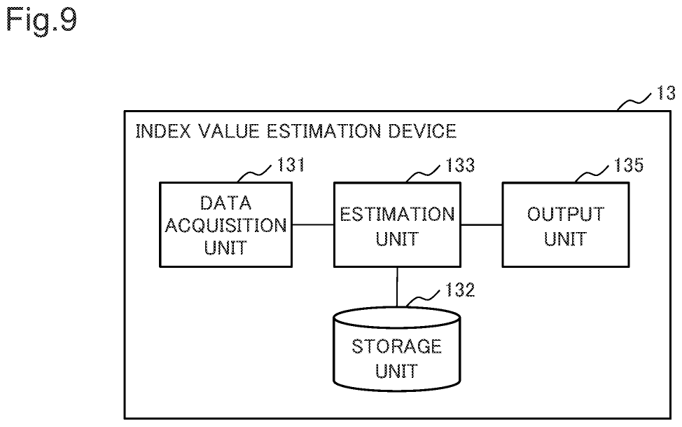

FIG. 9 is a block diagram illustrating an example of a configuration of the index value estimation device 13. The index value estimation device 13 includes a data acquisition unit 131, a storage unit 132, an estimation unit 133, and an output unit 135.

The data acquisition unit 131 receives the feature amount data from the measurement device 10. The data acquisition unit 131 outputs the received feature amount data to the estimation unit 133. The data acquisition unit 131 communicates with the transmission unit 127 of the measurement device 10 by a common communication method. The data acquisition unit 131 receives the feature amount data from the measurement device 10 via wireless communication. For example, the data acquisition unit 131 is configured to receive the feature amount data from the measurement device 10 via a wireless communication function (not illustrated) conforming to a standard such as Bluetooth (registered trademark) or WiFi (registered trademark). The communication function of the data acquisition unit 131 may conform to a standard other than Bluetooth (registered trademark) or WiFi (registered trademark). The data acquisition unit 131 may be configured to receive the feature amount data from the measurement device 10 via a wire such as a cable.

The storage unit 132 stores an estimation model that estimates an index value indicating the knee state using the feature amount data extracted from the gait waveform data. The storage unit 132 stores an estimation model that is trained in the relationship between the feature amount data regarding the knee flexion angles of the plurality of subjects and the index value indicating the knee state. For example, the storage unit 132 stores an estimation model that is trained for a plurality of subjects and that estimates a parameter regarding a knee flexion angle. Details of the parameter related to the knee flexion angle will be described later.

The estimation model may be stored in the storage unit 132 at the time of factory shipment of a product, calibration before the user uses the estimation system, or the like. For example, an estimation model stored in a storage device such as an external server may be used. In this case, the estimation model may be configured to be used via an interface (not illustrated) connected to the storage device.

The estimation unit 133 acquires the feature amount data from the data acquisition unit 131. The estimation unit 133 executes estimation of a parameter regarding a knee flexion angle as an index value indicating a knee state using the acquired feature amount data. The estimation unit 133 inputs the feature amount data to the estimation model stored in the storage unit 132. The estimation unit 133 outputs an estimation result related to the index value (parameter regarding the knee flexion angle) indicating the knee state output from the estimation model. In a case where an estimation model stored in an external storage device constructed in a cloud, a server, or the like is used, the estimation unit 133 is configured to use the estimation model via an interface (not illustrated) connected to the storage device.

The output unit 135 outputs the estimation result of the index value (parameter regarding the knee flexion angle) indicating the knee state by the estimation unit 133. For example, the output unit 135 displays the estimation result of the index value indicating the knee state on the screen of the mobile terminal of the subject (user). For example, the output unit 135 outputs the estimation result to an external system or the like that uses the estimation result. The use of the index value indicating the knee state output from the index value estimation device 13 is not particularly limited.

For example, the index value estimation device 13 is connected to an external system or the like constructed in a cloud or a server via a mobile terminal (not illustrated) carried by a subject (user). The mobile terminal (not illustrated) is a portable communication device. For example, the mobile terminal is a portable communication device having a communication function, such as a smartphone, a smart watch, or a mobile phone. For example, the index value estimation device 13 is connected to a mobile terminal via wireless communication. For example, the index value estimation device 13 is connected to a mobile terminal via a wireless communication function (not illustrated) conforming to a standard such as Bluetooth (registered trademark) or WiFi (registered trademark). The communication function of the index value estimation device 13 may conform to a standard other than Bluetooth (registered trademark) or WiFi (registered trademark). For example, the index value estimation device 13 may be connected to a mobile terminal via a wire such as a cable. The estimation result of the index value indicating the knee state may be used by an application installed in the mobile terminal. In this case, the mobile terminal executes processing using the estimation result by application software or the like installed in the mobile terminal.

### [Parameter Related to Knee Flexion Angle]

Next, the correlation between the parameter related to the knee flexion angle and the feature amount data will be described together with the verification result. Hereinafter, a verification example performed on 72 subjects (36 males and 36 females) will be described. In the following verification example, the correlation between the measured value and the estimation value of the parameter regarding the knee flexion angle in gait was verified. In the following verification, a subject wearing a smart apparel and a shoe on which the measurement device 10 is mounted was caused to make a round trip by walk twice on a straight path of 5 m. The measured value was obtained by measuring the knee bending joint angle of the subject wearing the smart apparel by a motion capture method. The prediction value is an estimation value estimated using sensor data measured by the measurement device 10 mounted in the shoe worn by the subject at the same time when the measured value was measured. Hereinafter, the correlation between the measured value and the estimation value is verified by a value of intraclass correlation coefficient (ICC).

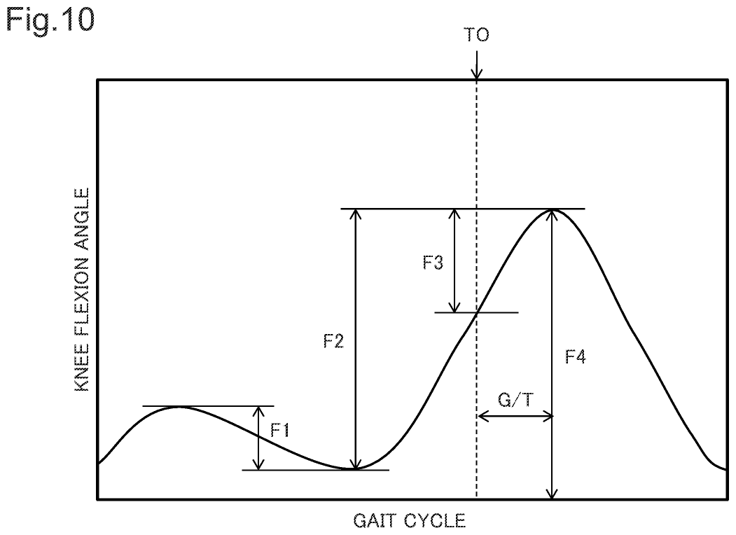

FIG. 10 is a graph illustrating an example of time series data of a knee flexion angle in one gait cycle. The horizontal axis of FIG. 10 is a gait cycle in which the timing of the heel contact is set as a starting point and the timing of the next heel contact is set as an end point. The gait cycle is normalized to 0 to 100%. The gait cycle is normalized in such a way that the stance phase is a 60% period and the swing phase is a 40% period.

As illustrated in FIG. 10, two peaks appear in the time series data of the knee flexion angle in one gait cycle. One valley appears between the two peaks. The first peak appears in a transition period from the initial stance period T1 to the mid-stance period T2. The timing of the first peak substantially coincides with the timing of the opposite toe off OTO. The second peak appears in a transition period from the initial swing period T5 of the mid-swing period T6. The timing of the second peak substantially coincides with the timing of the foot adjacent FA.

FIG. 10 illustrates a parameter related to the knee flexion angle as an example of the index value indicating the knee state. In the present example embodiment, an example of estimating a first angle parameter F1, a second angle parameter F2, a third angle parameter F3, a fourth angle parameter F4, a gait cycle parameter G, and a time parameter T as parameters related to the knee flexion angle will be described.

Non-Patent Literature 4 (S. R. Goldberg, et al., “Muscles that influence knee flexion velocity in double support: implications for stiff-knee gait”, Journal of Biomechanics, 37, (2004), pp. 1189-1196) describes Stiff-knee Gait, which is an event in which the knee flexion angle decreases during the gait in a cerebral palsy patient. Stiff-knee Gait is defined as “claudication exhibiting a decrease in knee flexion angle in the swing phase”. In recent years, the term Stiff-knee Gait is also used for abnormal gait caused by a knee joint disease. In Stiff-knee Gait, an increase in the risk of falling due to toe catching and a decrease in the gait speed/gait energy efficiency occur. In knee osteoarthritis, the knee flexion angle may decrease. The presence or absence of Stiff-knee Gait may be available as a parameter for determining a disease such as knee osteoarthritis. Non-Patent Literature 4 reports that, in a case where the bending speed of the knee at the toe off is insufficient, Stiff-knee Gait occurs, and there is a possibility that the knee flexion angle in the swing phase decreases.

The first angle parameter F1 is a value obtained by subtracting the knee flexion angle at the timing of the valley between the two peaks from the knee flexion angle at the timing of the first peak. In a case where there is a disease in the knee, the valley between the two peaks tends to be unclear. Therefore, in a case where there is a disease in the knee, the first angle parameter F1 decreases.

The second angle parameter F2 is a value obtained by subtracting the knee flexion angle at the timing of the valley between the two peaks from the knee flexion angle at the timing of the second peak. In a case where there is a disease in the knee, the valley between the two peaks tends to be unclear. Therefore, in a case where there is a disease in the knee, the second angle parameter F2 decreases.

The third angle parameter F3 is a value obtained by subtracting the knee flexion angle at the timing of the toe off from the knee flexion angle at the timing of the second peak. In a case where there is a disease in the knee, the knee flexion angle tends to decrease. Therefore, in a case where there is a disease in the knee, the third angle parameter F3 decreases.

The fourth angle parameter F4 is a knee flexion angle at the timing of the second peak. In a case where there is a disease in the knee, the knee flexion angle in the swing phase tends to decrease. Therefore, in a case where the knee has a disease, the fourth angle parameter F4 decreases.

The gait cycle parameter G is a temporal distance (gait cycle) from the timing of the toe off to the timing of the second peak. In a case where there is a disease in the knee, the knee speed at the timing of the toe off tends to decrease. Therefore, in a case where there is a disease in the knee, the gait cycle parameter G increases.

The time parameter T is a time from the timing of the toe off to the timing of the second peak. In a case where there is a disease in the knee, the knee speed at the timing of the toe off tends to decrease. Therefore, in a case where there is a disease in the knee, the time parameter T increases.

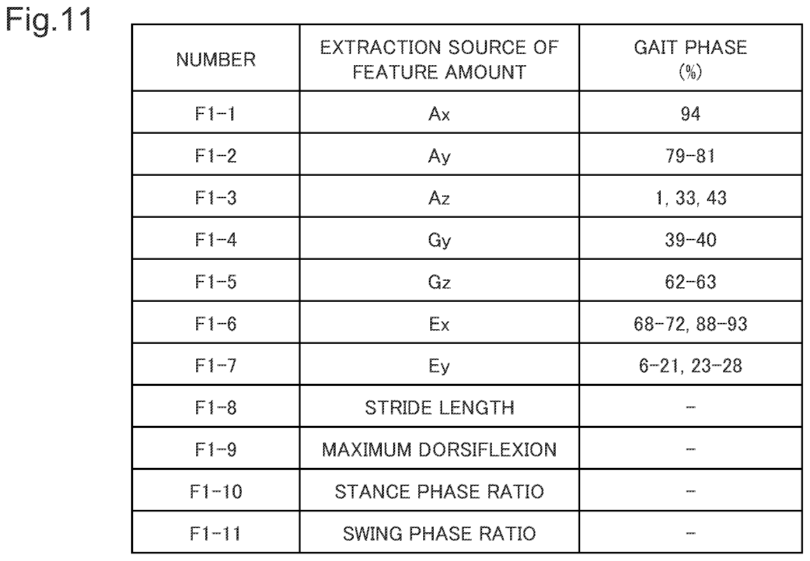

FIGS. 11 to 15 are correspondence tables summarizing feature amounts used for estimating parameters related to knee flexion angles. In the correspondence table of FIGS. 11 to 15, the feature amount number, the extraction source of the feature amount (the gait waveform data and the gait parameter), and the gait phase (%) included in the gait phase cluster are associated with each other. The feature amount used for the estimation of the parameter related to the knee flexion angle was selected based on the correlation between the measured value and the estimation value. The following feature amount used for the estimation of the parameter regarding the knee flexion angle is an example, and does not limit the feature amount used for the estimation of the parameter regarding the knee flexion angle.

FIG. 11 is a correspondence table summarizing feature amounts used to estimate the first angle parameter F1. The feature amounts F1-1 to 11 were used to estimate the first angle parameter F1. The feature amount F1-1 was extracted from the gait phase 94% of the gait waveform data Ax regarding the time series data of the left-right acceleration (X direction acceleration). The feature amount F1-2 was extracted from the section of the gait phase 79 to 81% of the gait waveform data Ay regarding the time series data of the acceleration in the traveling direction (Y direction acceleration). The feature amount F1-3 was extracted from the gait phases 1%, 33%, and 43% of the gait waveform data Az regarding the time series data of the vertical acceleration (Z direction acceleration). The feature amount F1-4 was extracted from the section of the gait phase 39 to 40% of the gait waveform data Gy regarding the time series data of the angular velocity in the coronal plane (around the Y axis). The feature amount F1-5 was extracted from the section of the gait phase 62 to 63% of the gait waveform data Gz regarding the time series data of the angular velocity in the horizontal plane (around the Z axis). The feature amount F1-6 was extracted from the sections of the gait phases 68 to 72% and 88 to 93% of the gait waveform data Ex regarding the time series data of the angle (posture angle) in the sagittal plane (around the X axis). The feature amount F1-7 was extracted from the sections of the gait phases 6 to 21% and 23 to 28% of the gait waveform data Ey regarding the time series data of the angle (posture angle) in the sagittal plane (around the Y axis). The feature amount F1-8 is a stride length included in the gait parameter. The feature amount F1-9 is a maximum value (maximum dorsiflexion) of the dorsiflexion included in the gait parameter. The feature amount F1-10 is a ratio of the stance phase in one gait cycle included in the gait parameter. The feature amount F1-11 is a ratio of the swing phase in one gait cycle included in the gait parameter. The intraclass correlation coefficient ICC between the measured value and the estimation value of the first angle parameter F1 was 0.4893.

For example, the estimation model that estimates the first angle parameter F1 outputs the first angle parameter F1 that is an index of an index value indicating the knee state according to the input of the feature amounts F1-1 to 11. Such an estimation model is generated by training using training data having the feature amounts F1-1 to 11 used for estimating the first angle parameter F1 as explanatory variables and the first angle parameter F1 as an objective variable.

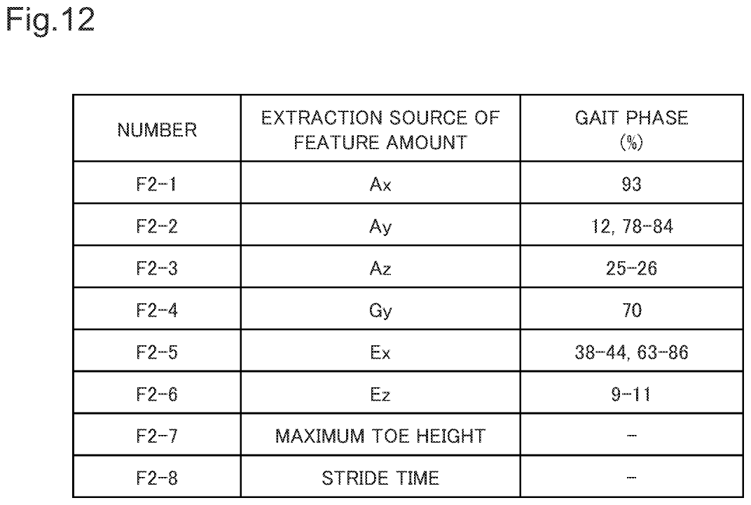

FIG. 12 is a correspondence table summarizing feature amounts used to estimate the second angle parameter F2. The feature amounts F2-1 to 8 were used to estimate the second angle parameter F2. The feature amount F2-1 was extracted from the gait phase 93% of the gait waveform data Ax regarding the time series data of the left-right acceleration (X direction acceleration). The feature amount F2-2 was extracted from the sections of the gait phases 12% and 78 to 84% of the gait waveform data Ay regarding the time series data of the acceleration in the traveling direction (Y direction acceleration). The feature amount F2-3 was extracted from the gait phase 25 to 26% of the gait waveform data Az regarding the time series data of the vertical acceleration (Z direction acceleration). The feature amount F2-4 was extracted from the section of the gait phase 70% of the gait waveform data Gy regarding the time series data of the angular velocity in the coronal plane (around the Y axis). The feature amount F2-5 was extracted from the sections of the gait phases 38 to 44% and 63 to 86% of the gait waveform data Ex regarding the time series data of the angle (posture angle) in the sagittal plane (around the X axis). The feature amount F2-6 was extracted from the section of the gait phase 9 to 11% of the gait waveform data Ez regarding the time series data of the angle (posture angle) in the horizontal plane (around the Z axis). The feature amount F2-7 is a maximum value (maximum toe height) of the toe height included in the gait parameter. The feature amount F2-8 is a stride time included in the gait parameter. The intraclass correlation coefficient ICC between the measured value and the estimation value of the second angle parameter F2 was 0.4732.

For example, the estimation model that estimates the second angle parameter F2 outputs the second angle parameter F2 that is an index of an index value indicating the knee state according to the input of the feature amounts F2-1 to 8. Such an estimation model is generated by training using training data having the feature amounts F2-1 to 8 used for estimating the second angle parameter F2 as explanatory variables and the second angle parameter F2 as an objective variable.

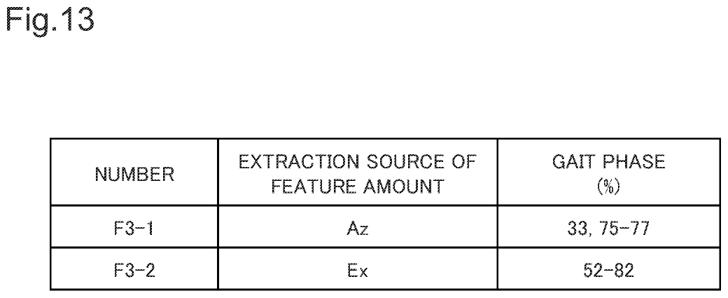

FIG. 13 is a correspondence table summarizing feature amounts used to estimate the third angle parameter F3. For the estimation of the third angle parameter F3, feature amounts F3-1 to 2 were used. The feature amount F3-1 was extracted from the gait phases 33% and 75 to 77% of the gait waveform data Az regarding the time series data of the vertical acceleration (Z direction acceleration). The feature amount F3-2 was extracted from the section of the gait phase 52 to 82% of the gait waveform data Ex regarding the time series data of the angle (posture angle) in the sagittal plane (around the X axis). The intraclass correlation coefficient ICC between the measured value and the estimation value of the third angle parameter F3 was 0.5944.

For example, the estimation model that estimates the third angle parameter F3 outputs the third angle parameter F3 that is an index of an index value indicating the knee state according to the input of the feature amounts F3-1 to 2. Such an estimation model is generated by training using training data having the feature amounts F3-1 to 2 used for estimating the third angle parameter F3 as explanatory variables and the third angle parameter F3 as an objective variable.

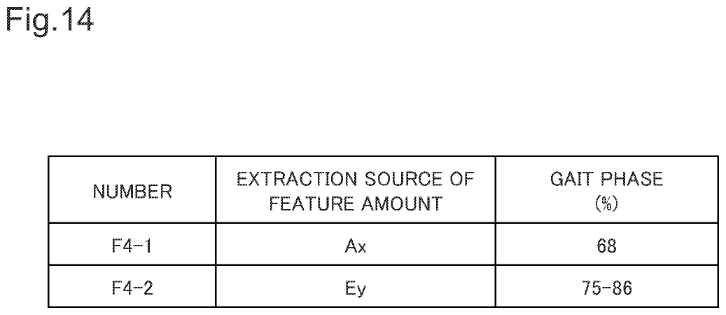

FIG. 14 is a correspondence table summarizing feature amounts used to estimate the fourth angle parameter F4. For the estimation of the fourth angle parameter F4, feature amounts F4-1 to 2 were used. The feature amount F4-1 was extracted from the gait phase 68% of the gait waveform data Ax regarding the time series data of the left-right acceleration (X direction acceleration). The feature amount F4-2 was extracted from the section of the gait phase 75 to 86% of the gait waveform data Ey regarding the time series data of the angle (posture angle) in the sagittal plane (around the Y axis). The intraclass correlation coefficient ICC between the measured value and the estimation value of the fourth angle parameter F4 was 0.3345.

For example, the estimation model that estimates the fourth angle parameter F4 outputs the fourth angle parameter F4 that is an index of an index value indicating the knee state according to the input of the feature amount F4-1 to 2. Such an estimation model is generated by training using training data having the feature amounts F4-1 to 2 used for estimating the fourth angle parameter F4 as explanatory variables and the fourth angle parameter F4 as an objective variable.

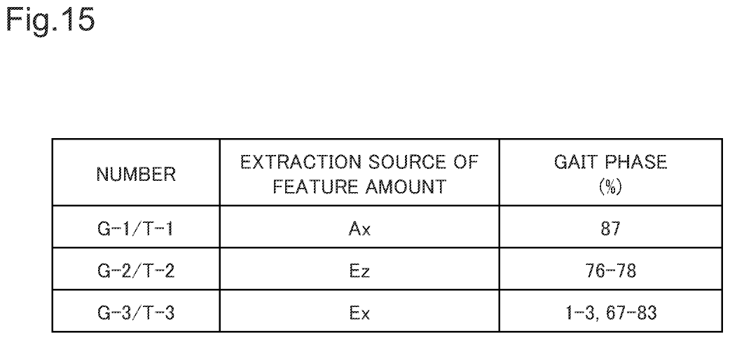

FIG. 15 is a correspondence table summarizing feature amounts used for estimating the gait cycle parameter G and the time parameter T. The feature amounts G-1 to 3 were used to estimate the gait cycle parameter G. The feature amounts T-1 to 3 were used to estimate the time parameter T. The feature amount G-1 and the feature amount T-1 were extracted from the gait phase 87% of the gait waveform data Ax regarding the time series data of the left-right acceleration (X direction acceleration). The feature amount G-2 and the feature amount T-2 were extracted from the section of the gait phase 76 to 78% of the gait waveform data Ez regarding the time series data of the angle (posture angle) in the horizontal plane (around the Z axis). The feature amount G-3 and the feature amount T-3 were extracted from the sections of the gait phases 1 to 3% and 67 to 83% of the gait waveform data Ex regarding the time series data of the angle (posture angle) in the sagittal plane (around the X axis). The intraclass correlation coefficient ICC between the measured value and the estimation value of the gait cycle parameter G was 0.4818. The intraclass correlation coefficient ICC between the measured value and the estimation value of the time parameter T was 0.7122.

For example, the estimation model that estimates the gait cycle parameter G outputs the gait cycle parameter G, that is an index of an index value indicating the knee state, according to the input of the feature amounts G-1 to 3. Such an estimation model is generated by training using training data having the feature amounts G-1 to 3 used for estimating the gait cycle parameter G as explanatory variables and the gait cycle parameter G as an objective variable.

For example, the estimation model that estimates the time parameter T outputs the time parameter T that is an index of an index value indicating the knee state according to the input of the feature amounts T-1 to 3. Such an estimation model is generated by training using training data having the feature amounts T-1 to 3 used for estimating the time parameter T as explanatory variables and the time parameter T as an objective variable.

The estimation result of the estimation model is not limited as long as the estimation result regarding the parameter regarding the knee flexion angle is output according to the input of the feature amount data. For example, the storage unit 132 stores an estimation model that estimates a parameter related to a knee flexion angle using a multiple regression prediction method. For example, the storage unit 132 stores coefficients (weights) to be integrated into individual pieces of feature amount data. The coefficients (weights) stored in the storage unit 132 are integrated into the feature amount data with which they are associated. The sum of the feature amount data obtained by integrating the coefficients (weights) corresponds to the parameter related to the knee flexion angle.

(Operation)

Next, an example of an operation of the estimation system 1 will be described with reference to the drawings. The measurement device 10 and the index value estimation device 13 included in the estimation system 1 will be individually described. With respect to the measurement device 10, the operation of the feature amount data generation unit 12 included in the measurement device 10 will be described.

### [Measurement Device]

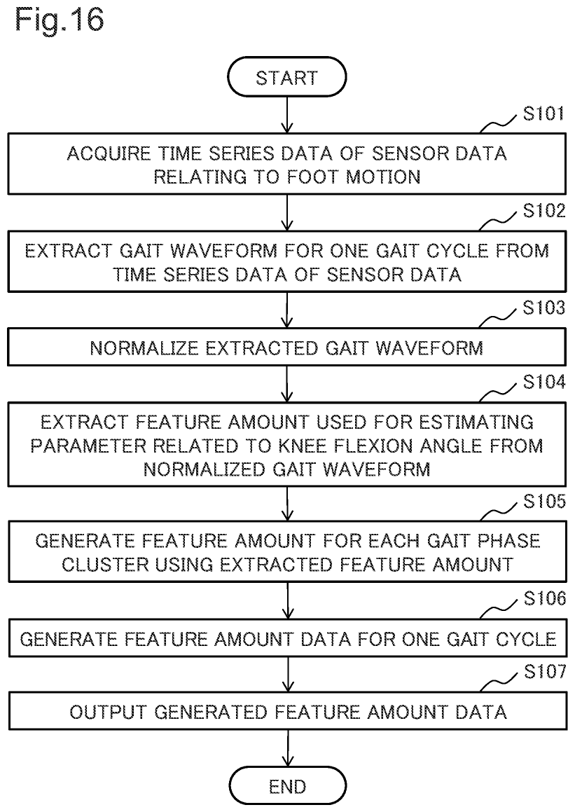

FIG. 16 is a flowchart for explaining the operation of the feature amount data generation unit 12 included in the measurement device 10. In the description along the flowchart of FIG. 16, the feature amount data generation unit 12 will be described as an operation subject.

In FIG. 16, first, the feature amount data generation unit 12 acquires time series data of sensor data related to the motion of the foot (step S101).

Next, the feature amount data generation unit 12 extracts gait waveform data for one gait cycle from the time series data of the sensor data (step S102). The feature amount data generation unit 12 detects the heel contact and the toe off from the time series data of the sensor data. The feature amount data generation unit 12 extracts time series data of a section between consecutive heel contacts as gait waveform data for one gait cycle.

Next, the feature amount data generation unit 12 normalizes the extracted gait waveform data for one gait cycle (step S103). The feature amount data generation unit 12 normalizes the gait waveform data for one gait cycle to a gait cycle of 0 to 100% (first normalization). Furthermore, the feature amount data generation unit 12 normalizes the ratio of the stance phase to the swing phase in the gait waveform data subjected to the first normalization for one gait cycle to 60:40 (second normalization).

Next, the feature amount data generation unit 12 extracts a feature amount from the gait phase used for estimating the parameter regarding the knee flexion angle from the normalized gait waveform (step S104). The feature amount data generation unit 12 extracts a feature amount input to an estimation model constructed in advance.

Next, the feature amount data generation unit 12 generates a feature amount for each gait phase cluster using the extracted feature amount (step S105).

Next, the feature amount data generation unit 12 integrates the feature amounts for respective gait phase clusters to generate feature amount data for one gait cycle (step S106).

Next, the feature amount data generation unit 12 outputs the generated feature amount data to the index value estimation device 13 (step S107).

### [Index Value Estimation Device]

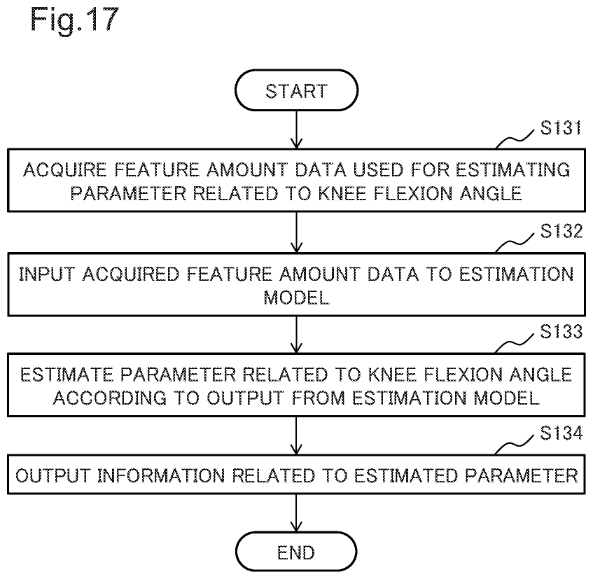

FIG. 17 is a flowchart for explaining the operation of the index value estimation device 13. In the description along the flowchart of FIG. 17, the index value estimation device 13 will be described as an operation subject.

In FIG. 17, first, the index value estimation device 13 acquires feature amount data used for estimating a parameter regarding a knee flexion angle (step S131).

Next, the index value estimation device 13 inputs the acquired feature amount data to an estimation model that estimates a parameter regarding a knee flexion angle (step S132).

Next, the index value estimation device 13 estimates a parameter related to the knee flexion angle of the user according to the output (estimation value) from the estimation model (step S133).

Next, the index value estimation device 13 outputs information about the estimated parameter (step S134). For example, the parameter related to the knee flexion angle is output to a terminal device (not illustrated) carried by the user. For example, the parameter related to the knee flexion angle is output to a system that executes processing using the parameter.

### Application Example

Next, an application example according to the present example embodiment will be described with reference to the drawings. In the following application example, an example in which the function of the index value estimation device 13 installed in the mobile terminal carried by the user estimates the information about the index value indicating the knee state using the feature amount data measured by the measurement device 10 disposed in the shoe 100 will be described.

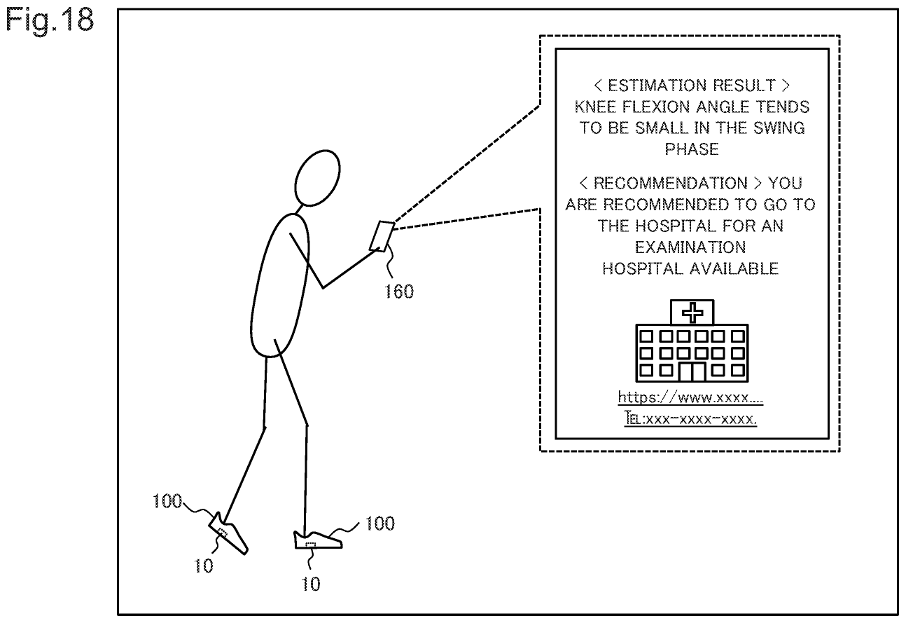

FIG. 18 is a conceptual diagram illustrating an example in which the estimation result by the index value estimation device 13 is displayed on the screen of a mobile terminal 160 carried by the user who walks while wearing the shoe 100 in which the measurement device 10 is disposed. FIG. 18 is an example in which information according to the estimation result of the parameter, regarding the knee flexion angle, estimated using the feature amount data according to the sensor data measured while the user is walking is displayed on the screen of the mobile terminal 160.

FIG. 18 is an example in which information according to the estimation result of the parameter regarding the knee flexion angle, which is the index value indicating the knee state, is displayed on the screen of the mobile terminal 160. In the example of FIG. 18, information of “Knee flexion angle tends to be small in the swing phase” is displayed on the display unit of the mobile terminal 160 according to the estimation result. In the example of FIG. 18, recommendation information of “You are recommended to go to the hospital for an examination” is displayed on the display unit of the mobile terminal 160 according to the estimation value of the parameter regarding the knee flexion angle which is an index value indicating the knee state. For example, a link destination to a site or a telephone number of a hospital at which it is possible to seek medical advice may be displayed on the screen of the mobile terminal 160. The user who has confirmed the information displayed on the display unit of the mobile terminal 160 goes to a hospital for an examination of a knee disease according to the recommendation information.

As described above, the estimation system of the present example embodiment includes a measurement device and an index value estimation device. The measurement device includes a sensor and a feature amount data generation unit. The sensor includes an acceleration sensor and an angular velocity sensor. The sensor measures a spatial acceleration with an acceleration sensor. The sensor measures a spatial angular velocity with an angular velocity sensor. The sensor uses the measured spatial acceleration and spatial angular velocity to generate sensor data related to the motion of the foot. The sensor outputs the generated sensor data to the feature amount data generation unit. The feature amount data generation device acquires time series data of sensor data related to a motion of a foot. The feature amount data generation device extracts gait waveform data for one gait cycle from the time series data of the sensor data. The feature amount data generation device normalizes the extracted gait waveform data. The feature amount data generation device extracts, from the normalized gait waveform data, a feature amount used for estimating an index value indicating a knee state from a gait phase cluster constituted by at least one temporally continuous gait phase. The feature amount data generation device generates feature amount data including the extracted feature amount. The feature amount data generation device outputs the generated feature amount data.

The index value estimation device includes a data acquisition unit, a storage unit, an estimation unit, and an output unit. A data acquisition unit acquires feature amount data including a feature amount that is extracted from sensor data related to a motion of a foot of a user and that is used for estimating an index value indicating a knee state of the user. The storage unit stores an estimation model that outputs an index value according to an input of feature amount data. The storage unit stores an estimation model that estimates a parameter related to a knee flexion angle as an index value indicating a knee state. The estimation unit estimates an output obtained by inputting the acquired feature amount data to the estimation model as an index value indicating the knee state of the user. The output unit outputs information related to the estimated index value indicating the knee state of the user. The estimation unit estimates a parameter regarding the knee flexion angle obtained by inputting the feature amount data acquired regarding the user to the estimation model as an index value indicating the knee state of the user.

The estimation system of the present example embodiment estimates an index value indicating the knee state of the user using a feature amount extracted from sensor data related to the motion of the foot of the user. Therefore, according to the present example embodiment, the parameter regarding the knee flexion angle can be appropriately estimated as the index value indicating the knee state in daily life without using a dedicated instrument for measuring the index value indicating the knee state.

In daily gait, the knee has an important function. Knee related diseases such as knee osteoarthritis can cause pain due to arthritis and the like, and can be a factor that adversely affects quality of life (QoL) of daily life. Early detection and prevention are important for these diseases. However, in order to diagnose these diseases, measurement by a specialized device or diagnosis by an expert is required. Therefore, it is difficult to detect/prevent these diseases early in daily life. According to the method of the present example embodiment, the parameter regarding the knee flexion angle can be appropriately estimated using the sensor data measured in daily life. In a case where the value of the estimated parameter deviates significantly from the healthy range, there may be a disease in the knee. The parameter obtained by the method of the present example embodiment can be used as auxiliary information for diagnosing a knee disease. That is, according to the method of the present example embodiment, it is possible to estimate an index value for early detection/prevention of a knee disease in daily life.

In an aspect of the present example embodiment, the storage unit stores an estimation model generated by training using training data having feature amounts obtained in verification regarding gait of a plurality of subjects as explanatory variables and a measured value of an index value actually measured in verification regarding gait of the plurality of subjects as an objective variable. The estimation unit estimates an output obtained by inputting the feature amount data acquired regarding the user to the estimation model as an index value indicating the knee state of the user. According to the present aspect, the index value indicating the knee state can be estimated from the statistical viewpoint by using the estimation model that is trained in the feature amount obtained by the verification on the plurality of subjects and the measured value of the index value.

In an aspect of the present example embodiment, the storage unit stores an estimation model that estimates a parameter regarding a knee flexion angle related to two peaks appearing in time series data of a knee flexion angle for one gait cycle. The estimation unit inputs the feature amount data acquired according to the gait of the user to the estimation model, and estimates the index value indicating the knee state of the user according to the index value of the user output from the estimation model. In the present aspect, the parameter regarding the knee flexion angle is estimated in association with the peak appearing in the time series data of the knee flexion angle. Therefore, according to the present aspect, it is possible to estimate the index value in which the motion of the knee during gait is more reflected.

In an aspect of the present example embodiment, the storage unit stores an estimation model that estimates a parameter related to a knee flexion angle including a temporal relationship between a timing of a peak appearing in a swing phase of two peaks appearing in time series data of a knee flexion angle for one gait cycle and a timing of a toe off. The estimation unit inputs the feature amount data acquired according to the gait of the user to the estimation model, and estimates the index value indicating the knee state of the user according to the index value of the user output from the estimation model. In the present aspect, the temporal relationship between the timing of the peak appearing in the swing phase of the time series data of the knee flexion angle and the timing of the toe off is estimated as the parameter regarding the knee flexion angle. Therefore, according to the present aspect, it is possible to estimate the index value in which the motion of the knee in the swing phase is more reflected.

### Second Example Embodiment

Next, an estimation system according to a second example embodiment will be described with reference to the drawings. In the present example embodiment, an example of estimating the cost indicating smoothness of the knee motion as the index value indicating the knee state will be described. In the present example embodiment, as the cost indicating smoothness of the knee motion, an example of estimating angular jerk cost (AJC) corresponding to a value obtained by integrating the square value of the angular jerk, which is the third derivative of the knee bending angle, in a specific period included in the gait cycle will be described.

(Configuration)

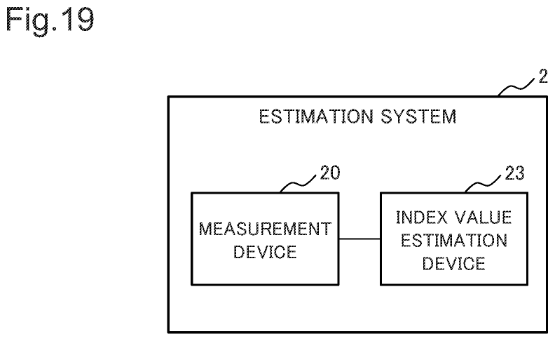

FIG. 19 is a block diagram illustrating an example of a configuration of an estimation system 2 according to the present example embodiment. The estimation system 2 includes a measurement device 20 and an index value estimation device 23. The measurement device 20 has the similar configuration as the measurement device 10 of the first example embodiment. The index value estimation device 23 has the similar configuration as the index value estimation device 13 of the first example embodiment. The measurement device 20 and the index value estimation device 23 are different from those of the first example embodiment in the extraction source of the feature amount used to estimate the index value indicating the knee state. The detailed configuration of the measurement device 20 and the index value estimation device 23 will not be described below.

The interpretation of the evaluation of the angular jerk is roughly divided into two. One interpretation is that the value of the angular jerk increases when the muscle strength is greatly exerted. Another interpretation is that the value of the angular jerk increases when smoothness of the motion decreases. A subject who has developed knee osteoarthritis has difficulty in appropriate kinematic response in the initial stance period, due to function of the knee joint, gait disorder, and the like due to factors such as knee pain and restriction of a range of motion. It is presumed that such a subject takes a measure to reduce a change in angular acceleration of the knee joint by reducing the floor reaction force and ensure smoothness of the motion to avoid the knee pain. In the present example embodiment, it is assumed that smoothness of the motion increases and the angular jerk decreases according to the compensation operation for avoiding the knee pain. Typically, the motion of the knee angle is not a constant acceleration motion. However, when the compensation operation for alleviating the knee pain is taken, the motion of the knee angle tends to be close to the constant acceleration motion.

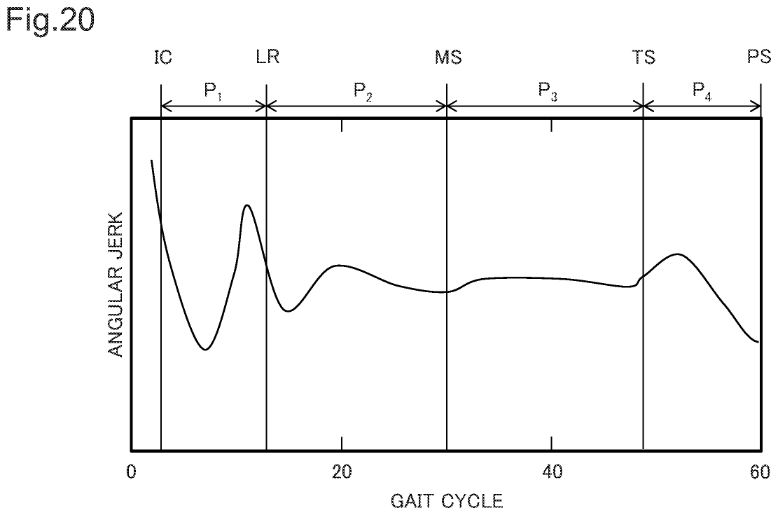

FIG. 20 is a graph illustrating an example of time series data of angular jerk. In the present example embodiment, AJC in each of a plurality of target sections included in a section (stance phase) of 0 to 60% of one gait cycle is estimated. In the present example embodiment, the AJC is estimated for each of a first section P₁, a second section P₂, a third section P₃, and a fourth section P₄ included in the stance phase. The first section P₁ is a section from an initial contact (IC) to a load reaction period (LR). The initial contact IC is a timing immediately after the heel contact HC. The load reaction period LR is a timing when the gait cycle is about 15%. The second section P₂ is a section from the load reaction period LR to a mid-stance MS. The mid-stance MS is a timing of transition from the mid-stance period T2 to the terminal stance period T3. The mid-stance MS is the central timing of the stance phase. The third section P₃ is a section from the mid-stance MS to a terminal stance TS. The terminal stance TS is a timing of transition from the terminal stance period T3 to the pre-swing period T4.

As in the first example embodiment, the measurement device 20 is mounted in the shoe of the subject. The measurement device 20 measures sensor data including acceleration (spatial acceleration) in three axis directions and angular velocities (spatial angular velocity) around three axes. The measurement device 20 normalizes the measured sensor data and extracts time series data (also referred to as gait waveform data) for one gait cycle. The measurement device 20 extracts a feature amount used for estimating the AJC from the gait waveform data. The measurement device 20 extracts a feature amount for each of the first section P₁, the second section P₂, the third section P₃, and the fourth section P₄. For example, the measurement device 20 extracts the feature amount of each of the first section P₁, the second section P₂, the third section P₃, and the fourth section P₄ from the first section P₁, the second section P₂, the third section P₃, and the fourth section P₄. The measurement device 20 generates feature amount data for each gait phase cluster using the extracted feature amount. The measurement device 20 transmits the generated feature amount data of the gait phase cluster to the index value estimation device 23 using the feature amount data.

The index value estimation device 23 receives the feature amount data from the measurement device 20. The index value estimation device 23 communicates with the measurement device 20 by a common communication method. The index value estimation device 23 stores an estimation model that estimates the AJC using the feature amount data extracted from the gait waveform data. The index value estimation device 23 stores an estimation model that is trained in the relationship between the feature amount data regarding the AJC of the plurality of subjects and the AJC. For example, the index value estimation device 23 stores an estimation model that is trained for a plurality of subjects and that estimates the AJC.

The index value estimation device 23 estimates the AJC as an index value indicating the knee state using the acquired feature amount data. The index value estimation device 23 inputs the feature amount data to the stored estimation model. The index value estimation device 23 outputs an estimation result according to the index value (AJC) indicating the knee state output from the estimation model. In a case where an estimation model stored in an external storage device constructed in a cloud, a server, or the like is used, the index value estimation device 23 is configured to use the estimation model via an interface (not illustrated) connected to the storage device.

The index value estimation device 23 outputs an estimation result of an index value (AJC) indicating a knee state. For example, the index value estimation device 23 displays the estimation result of the index value indicating the knee state on the screen of the mobile terminal of the subject (user). For example, the index value estimation device 23 outputs the estimation result to an external system or the like that uses the estimation result. The use of the index value indicating the knee state output from the index value estimation device 23 is not particularly limited.

Next, the correlation between the AJC estimated in each section and the feature amount data will be described together with the verification result. Hereinafter, as in the first example embodiment, a verification example performed on 72 (36 males and 36 females) subjects will be described. The feature amount used for the estimation of the AJC was selected based on the correlation between the measured value and the estimation value. The intraclass correlation coefficient ICC between the measured value and the estimation value in the first section P₁ was 0.2453. The intraclass correlation coefficient ICC between the measured value and the estimation value in the second section P₂ was 0.4418. The intraclass correlation coefficient ICC between the measured value and the estimation value in the third section P₃ was 0.6114. The intraclass correlation coefficient ICC between the measured value and the estimation value in the fourth section P₄ was 0.6185. The intraclass correlation coefficient ICC between the measured value and the estimation value was different depending on the sections. In the first section P₁, the movement of the measurement device 20 is complicated, and noise is likely to be included in the sensor data. As a result, it is estimated that the intraclass correlation coefficient ICC between the measured value and the estimation value decreased. On the other hand, in the third section P₃ and the fourth section P₄, it is estimated that the movement of the measurement device 20 is stabilized, and the intraclass correlation coefficient ICC between the measured value and the estimation value was relatively good.

(Operation)

Next, an example of an operation of the estimation system 2 will be described with reference to the drawings. The measurement device 20 and the index value estimation device 23 included in the estimation system 2 will be individually described.

### [Measurement Device]

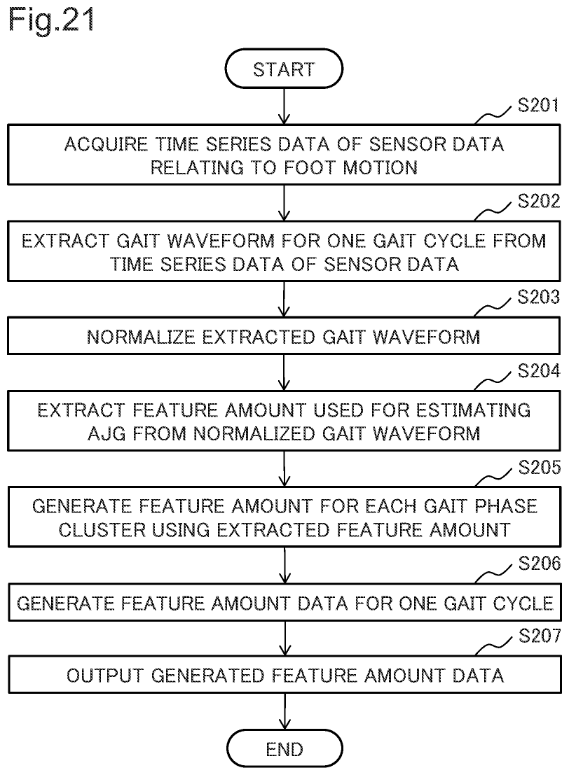

FIG. 21 is a flowchart for explaining the operation of the measurement device 20. In the description along the flowchart of FIG. 21, the measurement device 20 will be described as an operation subject.

In FIG. 21, first, the measurement device 20 acquires time series data of sensor data related to the motion of the foot (step S201).

Next, the measurement device 20 extracts gait waveform data for one gait cycle from the time series data of the sensor data (step S202). The measurement device 20 detects the heel contact and the toe off from the time series data of the sensor data. The measurement device 20 extracts time series data of a section between consecutive heel contacts as gait waveform data for one gait cycle.

Next, the measurement device 20 normalizes the extracted gait waveform data for one gait cycle (step S203). The measurement device 20 normalizes the gait waveform data for one gait cycle to a gait cycle of 0 to 100% (first normalization). Furthermore, the measurement device 20 normalizes the ratio of the stance phase to the swing phase in the gait waveform data subjected to the first normalization for one gait cycle to 60:40 (second normalization).

Next, the measurement device 20 extracts a feature amount from a gait phase used for estimating the AJC from the normalized gait waveform (step S204). The measurement device 20 extracts a feature amount input to an estimation model constructed in advance.

Next, the measurement device 20 generates a feature amount for each gait phase cluster using the extracted feature amount (step S205).

Next, the measurement device 20 integrates the feature amounts for respective gait phase clusters to generate feature amount data for one gait cycle (step S206).

Next, the measurement device 20 outputs the generated feature amount data to the index value estimation device 23 (step S207).

### [Index Value Estimation Device]

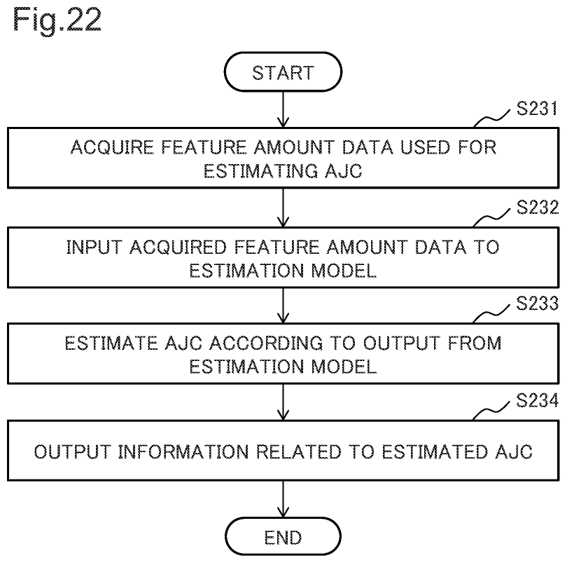

FIG. 22 is a flowchart for explaining the operation of the index value estimation device 23. In the description along the flowchart of FIG. 22, the index value estimation device 23 will be described as an operation subject.

In FIG. 22, first, the index value estimation device 23 acquires feature amount data used for estimating the AJC (step S231).

Next, the index value estimation device 23 inputs the acquired feature amount data to an estimation model that estimates the AJC (step S232).

Next, the index value estimation device 23 estimates the AJC according to the output (estimation value) from the estimation model (step S233).

Next, the index value estimation device 23 outputs information about the estimated AJC (step S234). For example, the AJC is output to a terminal device (not illustrated) carried by the user. For example, the AJC is output to a system that executes processing using a parameter.

### Application Example

Next, an application example according to the present example embodiment will be described with reference to the drawings. In the following application example, an example in which the function of the index value estimation device 23 installed in the mobile terminal carried by the user estimates the information about the index value indicating the knee state using the feature amount data measured by the measurement device 20 disposed in the shoe 200 will be described.

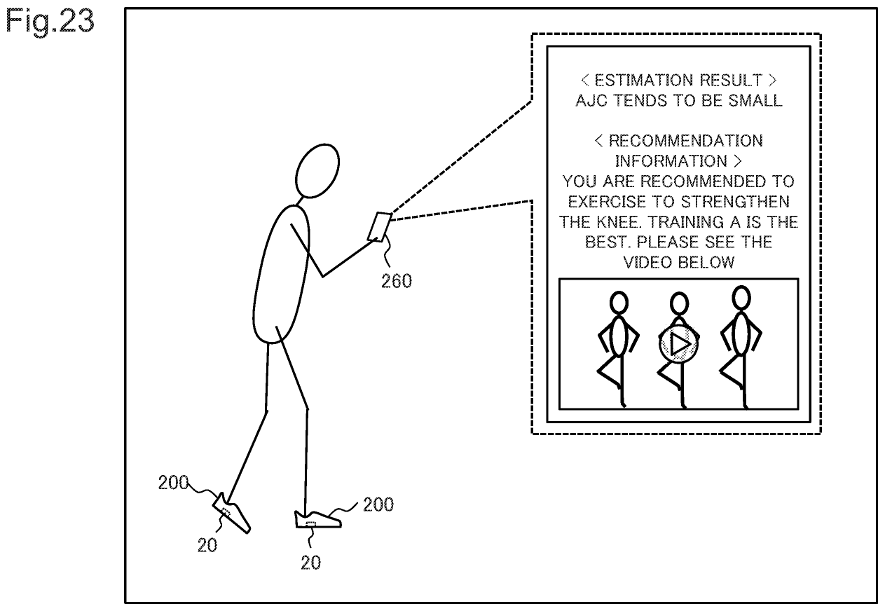

FIG. 23 is a conceptual diagram illustrating an example in which the estimation result by the index value estimation device 23 is displayed on the screen of a mobile terminal 260 carried by the user gait while wearing the shoe 200 on which the measurement device 20 is disposed. FIG. 23 is an example in which information related to the estimation result of the AJC estimated using the feature amount data related to the sensor data measured while the user is walking is displayed on the screen of the mobile terminal 260.

FIG. 23 illustrates an example in which information related to the estimation result of the AJC, which is an index value indicating the knee state, is displayed on the screen of the mobile terminal 260. In the example of FIG. 23, information of “AJC tends to be small” is displayed on the display unit of the mobile terminal 260 according to the estimation result. In the example of FIG. 23, according to the estimation value of the AJC which is an index value indicating the knee state, recommendation information according to the AJC estimation result of “You are recommended to exercise to strengthen the knee. Training A is the best. Please see the video below” is displayed on the display unit of the mobile terminal 260. The user who has confirmed the information displayed on the display unit of the mobile terminal 260 can practice training leading to improvement in the knee state by exercising with reference to the video of the training A according to the recommendation information.

As described above, the estimation system of the present example embodiment includes a measurement device and an index value estimation device. The measurement device includes a sensor and a feature amount data generation unit. The sensor includes an acceleration sensor and an angular velocity sensor. The sensor measures a spatial acceleration with an acceleration sensor. The sensor measures a spatial angular velocity with an angular velocity sensor. The sensor uses the measured spatial acceleration and spatial angular velocity to generate sensor data related to the motion of the foot. The sensor outputs the generated sensor data to the feature amount data generation unit. The feature amount data generation device acquires time series data of sensor data related to a motion of a foot. The feature amount data generation device extracts gait waveform data for one gait cycle from the time series data of the sensor data. The feature amount data generation device normalizes the extracted gait waveform data. The feature amount data generation device extracts, from the normalized gait waveform data, a feature amount used for estimating an index value indicating a knee state from a gait phase cluster constituted by at least one temporally continuous gait phase. The feature amount data generation device generates feature amount data including the extracted feature amount. The feature amount data generation device outputs the generated feature amount data.

The index value estimation device includes a data acquisition unit, a storage unit, an estimation unit, and an output unit. A data acquisition unit acquires feature amount data including a feature amount that is extracted from sensor data related to a motion of a foot of a user and that is used for estimating an index value indicating a knee state of the user. The storage unit stores an estimation model that outputs an index value according to an input of feature amount data. The storage unit stores an estimation model that estimates a cost indicating smoothness of the knee motion as an index value indicating the knee state. The estimation unit estimates an output obtained by inputting the acquired feature amount data to the estimation model as an index value indicating the knee state of the user. The output unit outputs information related to the estimated index value indicating the knee state of the user. The estimation unit estimates, as an index value indicating a knee state of the user, a cost indicating smoothness of the knee motion obtained by inputting the feature amount data acquired regarding the user to the estimation model.

The estimation system of the present example embodiment estimates an index value indicating the knee state of the user using a feature amount extracted from sensor data related to the motion of the foot of the user. Therefore, according to the present example embodiment, it is possible to appropriately estimate the cost indicating smoothness of the knee motion as the index value indicating the knee state in daily life without using a dedicated instrument for measuring the index value indicating the knee state.

According to the method of the present example embodiment, the cost indicating smoothness of the knee motion can be appropriately estimated using the sensor data measured in daily life. In a case where the estimated cost value deviates significantly from the healthy range, the knee may have a disease. The parameter obtained by the method of the present example embodiment can be used as auxiliary information for diagnosing a knee disease. That is, according to the method of the present example embodiment, it is possible to estimate an index value for early detection/prevention of a knee disease in daily life.

In an aspect of the present example embodiment, the storage unit stores an estimation model that estimates a cost indicating smoothness of the knee motion as an index value indicating the knee state for each of the plurality of sections included in the stance phase. The estimation unit estimates a cost indicating smoothness of the knee motion obtained by inputting the feature amount data acquired regarding the user to the estimation model for at least any one of the plurality of sections as an index value indicating the knee state of the user. In the present aspect, the cost indicating smoothness of the knee motion in the stance phase is estimated as the index value indicating the knee state. A person walking while enduring knee pain tends to move the knee smoothly in the stance phase. Therefore, according to the present aspect, the index value capable of detecting the user having the abnormality in the knee can be estimated.

### Third Example Embodiment

Next, an index value estimation device according to a third example embodiment will be described with reference to the drawings. The index value estimation device of the present example embodiment has a simplified configuration of the index value estimation device included in the estimation systems of the first and second example embodiments.

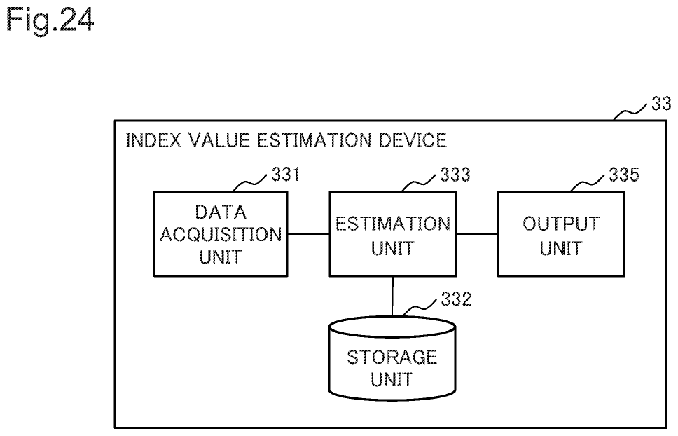

FIG. 24 is a block diagram illustrating an example of a configuration of an index value estimation device 33 according to the present example embodiment. The index value estimation device 33 includes a data acquisition unit 331, a storage unit 332, an estimation unit 333, and an output unit 335.

The data acquisition unit 331 acquires feature amount data including a feature amount that is extracted from sensor data related to a motion of a foot of a user and that is used for estimating an index value indicating a knee state of the user. The storage unit 332 stores an estimation model that outputs an index value according to the input of the feature amount data. The estimation unit 333 estimates an output obtained by inputting the acquired feature amount data to the estimation model as an index value indicating the knee state of the user. The output unit 335 outputs information related to the estimated index value indicating the knee state of the user.

As described above, in the present example embodiment, the index value indicating the knee state of the user is estimated using the feature amount extracted from the sensor data related to the motion of the foot of the user. Therefore, according to the present example embodiment, it is possible to appropriately estimate the index value indicating the knee state in daily life without using a dedicated instrument for measuring the index value indicating the knee state.

(Hardware)

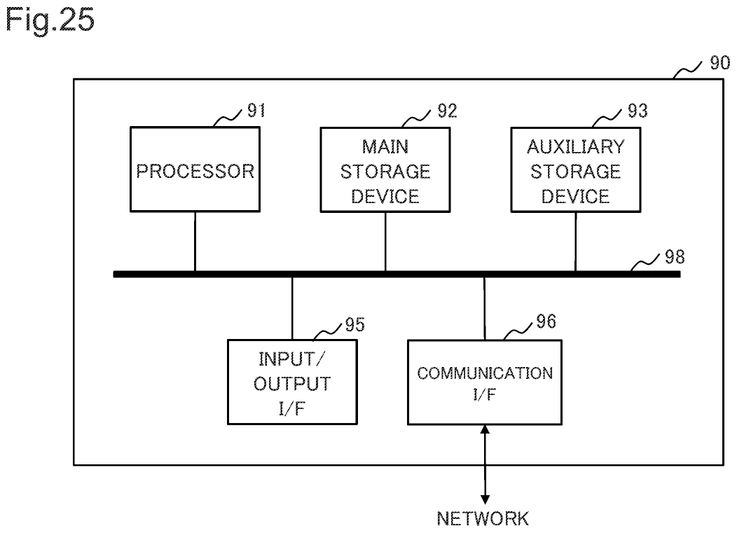

Regarding a hardware configuration that executes control and processing according to each example embodiment of the present disclosure, an information processing device 90 in FIG. 25 will be described as an example. The information processing device 90 in FIG. 25 is a configuration example for executing control and processing of each example embodiment, and does not limit the scope of the present disclosure.

As illustrated in FIG. 25, the information processing device 90 includes a processor 91, a main storage device 92, an auxiliary storage device 93, an input/output interface 95, and a communication interface 96. In FIG. 25, the interface is abbreviated as an interface (I/F). The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops the program stored in the auxiliary storage device 93 or the like in the main storage device 92. The processor 91 executes the program developed in the main storage device 92. In the present example embodiment, a software program installed in the information processing device 90 may be used. The processor 91 executes control and processing according to each example embodiment.

The main storage device 92 has an area in which a program is developed. A program stored in the auxiliary storage device 93 or the like is developed in the main storage device 92 by the processor 91. The main storage device 92 is achieved by, for example, a volatile memory such as a dynamic random access memory (DRAM). A nonvolatile memory such as a magnetoresistive random access memory (MRAM) may be configured and added as the main storage device 92.

The auxiliary storage device 93 stores various pieces of data such as programs. The auxiliary storage device 93 is achieved by a local disk such as a hard disk or a flash memory. Various pieces of data may be stored in the main storage device 92, and the auxiliary storage device 93 may be omitted.

The input/output interface 95 is an interface that connects the information processing device 90 with a peripheral device based on a standard or a specification. The communication interface 96 is an interface that connects to an external system or a device through a network such as the Internet or an intranet in accordance with a standard or a specification. The input/output interface 95 and the communication interface 96 may be shared as an interface connected to an external device.

An input device such as a keyboard, a mouse, or a touch panel may be connected to the information processing device 90 as necessary. These input devices are used to input of information and settings. In a case where the touch panel is used as the input device, the display screen of the display device may also serve as the interface of the input device. Data communication between the processor 91 and the input device may be mediated by the input/output interface 95.

The information processing device 90 may be provided with a display device that displays information. In a case where a display device is provided, the information processing device 90 preferably includes a display control device (not illustrated) that controls display of the display device. The display device may be connected to the information processing device 90 via the input/output interface 95.

The information processing device 90 may be provided with a drive device. The drive device mediates reading of data and a program from the recording medium, writing of a processing result of the information processing device 90 to the recording medium, and the like between the processor 91 and the recording medium (program recording medium). The drive device may be connected to the information processing device 90 via the input/output interface 95.

The above is an example of a hardware configuration for enabling control and processing according to each example embodiment of the present invention. The hardware configuration of FIG. 25 is an example of a hardware configuration for executing control and processing according to each example embodiment, and does not limit the scope of the present invention. A program for causing a computer to execute control and processing according to each example embodiment is also included in the scope of the present invention. A program recording medium in which the program according to each example embodiment is recorded is also included in the scope of the present invention. The recording medium can be achieved by, for example, an optical recording medium such as a compact disc (CD) or a digital versatile disc (DVD). The recording medium may be achieved by a semiconductor recording medium such as a Universal Serial Bus (USB) memory or a secure digital (SD) card. The recording medium may be achieved by a magnetic recording medium such as a flexible disk, or another recording medium. In a case where the program executed by the processor is recorded in the recording medium, the recording medium is a program recording medium.

The components of each example embodiment may be combined in any manner. The components each example embodiment may be achieved by software or may be achieved by a circuit.

The previous description of embodiments is provided to enable a person skilled in the art to make and use the present invention. Moreover, various modifications to these example embodiments will be readily apparent to those skilled in the art, and the generic principles and specific examples defined herein may be applied to other embodiments without the use of inventive faculty. Therefore, the present invention is not intended to be limited to the example embodiments described herein but is to be accorded the widest scope as defined by the limitations of the claims and equivalents.

Further, it is noted that the inventor's intent is to retain all equivalents of the claimed invention even if the claims are amended during prosecution.

What is claimed is:

1. An index value estimation device comprising:
a storage storing an estimation model that outputs an index value according to an input of feature amount data;
at least one memory storing instructions; and
at least one processor connected to the at least one memory and configured to execute the instructions to:
acquire feature amount data including a feature amount to be used for estimating an index value indicating a knee state of a user, the feature amount being extracted from sensor data related to a motion of a foot of the user;
estimate, as the index value indicating the knee state of the user, an output obtained by inputting the acquired feature amount data to the estimation model; and
output information related to the estimated index value indicating the knee state of the user.

2. The index value estimation device according to claim 1, wherein
the storage stores the estimation model generated by training using training data having, as an explanatory variable, a feature amount used for estimating the index value indicating a knee state extracted from the sensor data obtained in verification regarding a gait of each of a plurality of subjects, and having, as an objective variable, a measured value of the index value indicating a knee state actually measured in verification regarding a gait of each of the plurality of subjects, and wherein
the at least one processor is configured to execute the instructions to
estimate, as the index value indicating the knee state of the user, an output obtained by inputting the feature amount data acquired regarding the user to the estimation model.

3. The index value estimation device according to claim 2, wherein
the storage stores the estimation model that estimates a parameter related to a knee flexion angle as the index value indicating a knee state, and wherein
the at least one processor is configured to execute the instructions to
estimate, as the index value indicating the knee state of the user, a parameter related to the knee flexion angle obtained by inputting the feature amount data acquired regarding the user to the estimation model.

4. The index value estimation device according to claim 3, wherein
the storage stores the estimation model that estimates a parameter related to the knee flexion angle associated with two peaks appearing in time series data of the knee flexion angle for one gait cycle, and wherein
the at least one processor is configured to execute the instructions to
input the feature amount data acquired according to a gait of the user to the estimation model, and
estimate the index value indicating the knee state of the user according to the index value of the user output from the estimation model.

5. The index value estimation device according to claim 3, wherein
the storage stores the estimation model that estimates a parameter related to the knee flexion angle including a temporal relationship between a timing of a peak appearing in a swing phase of two peaks appearing in time series data of the knee flexion angle for one gait cycle and a timing of a toe off, and wherein
the at least one processor is configured to execute the instructions to
input the feature amount data acquired according to a gait of the user to the estimation model, and
estimate the index value indicating the knee state of the user according to the index value of the user output from the estimation model.

6. The index value estimation device according to claim 2, wherein
the storage stores the estimation model that estimates a cost indicating smoothness of a knee motion as the index value indicating a knee state, and wherein
the at least one processor is configured to execute the instructions to
estimate, as the index value indicating the knee state of the user, the cost indicating smoothness of a knee motion obtained by inputting the feature amount data acquired regarding the user to the estimation model.

7. The index value estimation device according to claim 6, wherein
the storage stores the estimation model that estimates the cost indicating smoothness of a knee motion as the index value indicating a knee state for each of a plurality of sections included in a stance phase, and wherein
the at least one processor is configured to execute the instructions to
estimate, as the index value indicating the knee state of the user, the cost indicating smoothness of the knee motion obtained by inputting the feature amount data acquired regarding the user to the estimation model for at least any one of the plurality of sections.

8. An estimation system comprising:
the index value estimation device according to claim 1; and
a measurement device installed in footwear of a user for whom an index value indicating a knee state is to be estimated, wherein
the measurement device includes
a sensor that measures a spatial acceleration and a spatial angular velocity, generates sensor data related to a motion of a foot using the measured spatial acceleration and the measured spatial angular velocity, and outputs the generated sensor data,
at least one memory storing instructions, and
at least one processor connected to the at least one memory, and wherein
the at least one processor is configured to execute the instructions to
acquire time series data of the sensor data including a feature of a gait,
extract gait waveform data for one gait cycle from the time series data of the sensor data,
normalize the extracted gait waveform data,
extract a feature amount used for estimating the index value indicating a knee state from the normalized gait waveform data from a gait phase cluster constituted by at least one temporally continuous gait phase,
generate feature amount data including the extracted feature amount, and
output the generated feature amount data to the index value estimation device.

9. An index value estimation method executed by a computer, the method comprising:
acquiring feature amount data including a feature amount to be used for estimating an index value indicating a knee state of a user, the feature amount being extracted from sensor data related to a motion of a foot of the user;
inputting the acquired feature amount data to an estimation model, the estimation model outputting an index value according to an input of the feature amount data;
estimating, as the index value indicating the knee state of the user, an output obtained by inputting the acquired feature amount data to the estimation model; and
outputting information related to the estimated index value indicating the knee state of the user.

10. A non-transitory recording medium storing a program for causing a computer to execute:
acquiring feature amount data including a feature amount to be used for estimating an index value indicating a knee state of a user, the feature amount being extracted from sensor data related to a motion of a foot of the user;
inputting the acquired feature amount data to an estimation model, the estimation model outputting an index value according to an input of the feature amount data;
estimating, as the index value indicating the knee state of the user, an output obtained by inputting the acquired feature amount data to the estimation model; and
outputting information related to the estimated index value indicating the knee state of the user.

11. The index value estimation device according to claim 1, wherein
the output is estimated by machine learning, and
the information is used for decision making to address the knee state of the user.
